(19) United States
(12) Patent Application Publication
(10) Pub. No.: US 2026/0274804 A1
(43) Pub. Date: Sep. 17, 2026

(54) PHENOLIC COMPOUNDS BLOCKING Nav 1.7 AND/OR Nav 1.8, PROCESSES FOR THE PREPARATION THEREOF, COMPOSITIONS, USES, METHODS OF TREATMENT USING SAME AND KITS

(71) Applicants: Eurofarma Laboratórios S.A., São Paulo (BR); Universidade Federal do Rio de Janeiro - UFRJ, Rio de Janeiro (BR)

(72) Inventors: GABRIELA BARREIRO, São Paulo (BR); DANILO PEREIRA DE SANT'ANA, São Paulo (BR); JÚLIA LAMMOGLIA MONTEIRO, São Paulo (BR); LUIS EDUARDO REINA GAMBA, São Paulo (BR); LÍDIA MOREIRA LIMA, Rio de Janeiro (BR); CARLOS ALBERTO MANSSOUR FRAGA, Rio de Janeiro (BR); ELIEZER JESUS DE LACERDA BARREIRO, Rio de Janeiro (BR)

(21) Appl. No.: 19/152,097

(22) PCT Filed: Jan. 26, 2024
(86) PCT No.: PCT/BR2024/050026

§ 371 Date: Jul. 30, 2025

Related U.S. Application Data
(60) Provisional application No. 63/482,211, filed on Jan. 30, 2023.

(51) Int. Cl. C07D 241/24; C07D 401/12

(52) U.S. Cl. CPC C07D 241/24; C07D 401/12

(57) ABSTRACT

The present invention relates to phenolic compounds blocking Nav 1.7 and/or Nav 1.8. More specifically, the present invention is related to phenols comprising Formula (I), in which the substituents R₁ to R₁₂ are selected independently of the groups defined in the specification, as well as their processes of preparation, compositions comprising at least one of these compounds, uses, treatment methods for treating or preventing pain-related pathologies and kits. The present invention belongs to the fields of medicinal chemistry, organic synthesis, as well as to the treatment of pain-related diseases.

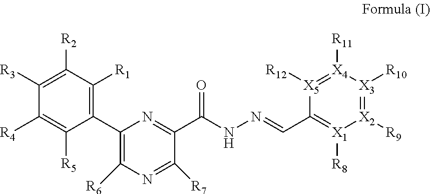

### FIELD OF INVENTION

The present invention refers to phenolic compounds blocking Nav 1.7 and/or Nav 1.8, processes for the preparation thereof, compositions containing these, uses, kits and treatment methods to treat or prevent pain-related pathologies. The present invention belongs to the fields of medicinal chemistry, organic synthesis, as well as to the treatment of pain-related diseases.

### BACKGROUND OF INVENTION

Physiological pain is an important protective mechanism designed to alert the body to real or potential injuries that can put the integrity thereof at risk. Broadly speaking, physiological pain can be classified as nociceptive pain and inflammatory pain. Nociceptive pain is characterized by having a high activation threshold, which remains until the stimulus that generated it is eliminated. Inflammatory pain, which arises as a response to tissue damage, is characterized by having a low activation threshold and is a consequence of the activity of molecular mediators of the inflammatory process in sensitizing nociceptors (Schaible. Langenbecks Arch. Surg. 2004, 389, 237). When these nociceptive processes remain in the absence of noxious stimuli or in response to non noxious stimuli, the protective and repair role of pain loses its functionality, configuring a maladaptive picture of neural plasticity and, as a consequence, a pathological state of chronic pain. Among the syndromes included in this classification, neuropathic pain has a high prevalence and impact today (Smith. Pain. 2020, 161, 1:S127; Cavalli. Int. J. Immunopathol. Pharmacol. 2019, 33:2058738419838383; Bouhassira. Rev Neurol (Paris). 2019, 175 (1-2):16; Scholz. Nature Neurosci., 2002, 5, 1062; Costigan. Annu. Rev. Neurosci., 2009, 32, 1).

Neuropathic pain is defined by the International Association for the Study of Pain (IASP) as pain initiated or caused by a primary dysfunction or injury in the central and/or peripheral nervous system (Dworkin. Clin. J. Pain, 2002, 18(6), 343). Central neuropathic pain comes from spinal cord injuries or central nervous system diseases such as multiple sclerosis or Parkinson's disease (Ducreux. Brain, 2006, 129, 963). Peripheral neuropathic pain, on the other hand, can be caused by trauma, metabolic disorders, chemical neurotoxicity, infection, or tumor invasion, among others. Among the most common syndromes of neuropathic pain are chemotherapy-induced neuropathic pain, complex regional pain, neuropathy related to viral infection, neuropathy secondary to tumor infiltration, diabetic neuropathy, phantom limb pain, postherpetic neuralgia, trigeminal neuralgia, and postsurgical neuralgia (Pak. Curr. Pain Headache Rep., 2018, 22(2), 9).

Currently, there is no specific treatment for the control of pathologies related to neuropathic pain, however, the first-line alternative consists of the use of opioid analgesics, and—as adjuvants—local anesthetics, anticonvulsants and antidepressants. However, the adverse effects and low efficacy drastically limit the use of these agents in the control of various pain-related pathologies (Kushnarev. Expert Opin. Investig. Drugs, 2020, 29(3), 259; Emery. Expert Opin. Ther. Targets, 2016, 20(8), 975).

Voltage-gated sodium channels (Nav) play a key role in the transmission of pain-related stimuli. These channels are activated in response to membrane depolarization, allowing the generation and propagation of action potentials in neurons (and other electrically excitable cells), by controlling the flow of sodium ions through the membranes. Structurally, voltage-gated sodium channels are heteromeric transmembrane proteins consisting of one a subunit and two β auxiliary subunits. The α subunit is organized into four homologous domains (I-IV), each with six transmembrane segments (S1-S6). The S4 segment of each domain is characterized by presenting a conserved region of arginine-residues, which act as sensors of the intra- and extracellular electrical environment of the neuron. This mechanism makes it possible to transform changes in the cellular electric field into specific conformational changes that, in turn, regulate the activation, deactivation and inactivation of voltage-gated sodium channels (Catterall. Nat. Chem. Biol., 2020, 16, 1314; Wisedchaisri. Cell., 2019, 178(4), 993; Clairfeuille. Science, 2019, 363, 1302).

In mammals, nine subunits α (Nav 1.1-Nav 1.9) and four auxiliary subunits β (β1-β4) have been identified. The α subunits can also be classified according to their susceptibility to blocking by tetrodotoxin (TTX), being classified as sensitive to tetrodotoxin (Nav 1.1, Nav 1.2, Nav 1.3, Nav 1.4, Nav 1.6 and Nav 1.7) or resistant to tetrodotoxin (Nav 1.5, Nav 1.8 and Nav 1.9) (Lera-Ruiz. J. Med. Chem., 2015, 58(18), 7093; Bagal. J. Med. Chem., 2013, 56(3), 593). Each of these a subunits has a different profile of expression and function, so that some of them are essential for the proper functioning of organs such as the heart and/or brain. Thus, the non-selective blocking of these channels is related to several types of adverse effects, such as migraine, epilepsy, paralysis and muscle and cardiac syndromes, among others (Bagal. J. Med. Chem., 2013, 56(3), 593; Bagal. Channels, 2015, 9(6), 360).

Broadly speaking, sodium channels are distributed mainly in the central and peripheral nervous system, in neurons and glia. Nav channels 1.1, 1.2, and 1.3 are primarily expressed in the brain. The Nav 1.4 and Nav 1.5 channels are found primarily in skeletal and cardiac muscles, respectively. Nav 1.6 channels are expressed in the central and peripheral nervous systems, while Nav 1.9 channels are selectively expressed in C-type nociceptive fibers in the dorsal root ganglion. On the other hand, the Nav 1.7 and Nav 1.8 channels are mainly found in the peripheral nervous system and are directly related to the processes of pain transmission (Law. Drug Discovery Today, 2019, 24(7), 1389; Bagal. Channels (Austin), 2015, 9(6), 360; Lera-Ruiz. J. Med. Chem., 2015, 58 (18), 7093).

Nav 1.7 sodium channels are expressed broadly in the olfactory epithelium, sympathetic ganglion, and dorsal root ganglion, predominantly in nociceptive fibers C and Ab. A large amount of evidence supports the important role of Nav 1.7 sodium channels in pain transmission processes. For example, gain-of-function related mutations in the gene (SCN9A), which encodes sodium channel Nav 1.7, are associated with extreme pain disorders such as congenital pain insensitivity, paroxysmal extreme pain disorder, and primary erythromelalgia. On the other hand, mutations related to loss of gene function (SCN9A) are related to congenital insensitivity to pain in individuals who, in general terms, are free of motor or cognitive impairment (Vetter. Pharmacology & Therapeutics, 2017, 172, 73; Ahuja. Science, 2015, 350(6267), 1491; Kingwell. Nat. Rev. Drug Discov., 2019, 18, 321; Safina. J. Med. Chem., 2021, 64, 2953; Luo. J. Med. Chem., 2019, 62, 831; Bankar. Cell Reports, 2018, 24, 3133).

Nav 1.8 sodium channels are most expressed in the peripheral nervous system, widely (but not exclusively) in C-type nociceptive fibers in the dorsal root ganglion. Recent evidence including elevated expression levels of Nav 1.8 in chronic pain states, data with Nav 1.8 knockout animals, and analgesic activity of Nav 1.8-specific desensitizing oligodeoxynucleotides, among others (Brown. Bioorg. Med. Chem., 2019, 27(1), 230; Payne. Br. J. Pharmacol., 2015, 172(10), 2654; Bagal. Med. Chem. Lett. 2015, 6(6) 650; Kort. J. Med. Chem. 2008, 51, 407; Zhang. Neuropharmacology, 2010, 59, 201 and 207), support the role of the sodium channel Nav 1.8 in the development and process of pain-related pathologies (Kingwell. Nat. Rev. Drug Discov., 2019, 18, 321; Law. Drug Discovery Today, 2019, 24(7), 1389; Bagal. Channels (Austin), 2015, 9(6), 360; Lera-Ruiz. J. Med. Chem., 2015, 58(18), 7093).

Thus, the voltage-gated sodium channels Nav 1.7 and Nav 1.8 are considered promising therapeutic targets for the treatment of neuropathic pain-related dysfunctions (Kornecook. J. Pharmacol. Exp. Ther., 2017, 362, 146; Kingwell. Nat. Rev. Drug Discov., 2019, 18, 321; Bagal. Channels (Austin), 2015, 9(6), 360; Lera-Ruiz. J. Med. Chem., 2015, 58(18), 7093; Deuis. Neuropharmacology, 2017, 127, 87 and 108; Kushnarev. Expert Opin. Investig. Drugs, 2020, 29(3), 259; McKerrall. Bioorganic & Medicinal Chemistry Lett., 2018, 28, 3141; Emery. Expert Opin. Ther. Targets, 2016, 20(8), 975; Bagal. Bioorganic & Medicinal Chemistry Lett., 2014, 24, 3690).

A large number of compounds have been described in the literature by their ability to act as blockers of Nav 1.7 and 1.8 sodium channels, however, they present a great structural diversity, which fact that does not allow the establishment of a common pharmacophoric group.

The patent literature contains several examples of compounds that act as sodium channel blockers. In particular, Nav 1.7 selective sodium channel blockers are described in U.S. Ser. No. 10/550,080, U.S. Pat. No. 9,765,029, and U.S. Ser. No. 10/000,475. Additionally, some documents describe selective blockers of Nav 1.8 sodium channels such as WO2020261114, WO2020092667, U.S. Pat. No. 9,163,042, WO2014120808, WO2014120815, WO2018213426, WO2019014352, WO2015006280, and U.S. Pat. No. 7,928,107. These documents reveal compounds with different structures from the present invention.

In addition, there are patent documents that describe dual Nav 1.7 and 1.8 blockers, including WO2018235851, U.S. Pat. No. 8,629,149, JP2017001991 that reveal, respectively, pyridyl amines, oxopiperazine derivatives and benzoxazolons. However, all these documents reveal compounds with structures and physicochemical characteristics different from the present invention.

In this context, it is advantageous to develop new alternatives of compounds that can act as Nav 1.7 and/or Nav 1.8 blockers that have adequate pharmacological action and, preferably, provide mitigated adverse effects. Therefore, the present invention refers to phenols as an alternative and/or complement to the treatment of pain-related diseases.

### SUMMARY OF THE INVENTION

The present invention discloses phenols with blocking activity of Nav 1.7 and/or 1.8 channels against pain-related pathologies, as well as related compositions, uses, kits, treatment methods and preparation processes.

The present invention refers to compound(s) of Formula (I):

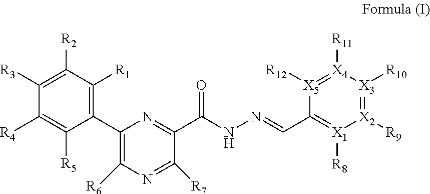

or a pharmaceutically acceptable salt, hydrate, solvate, ester and isomer thereof, wherein:
   one of X₁, X₂, X₃, X₄ and X₅ is nitrogen and the others are carbon, where when one of X₁, X₂, X₃, X₄ and X₅ is nitrogen, the corresponding R (R₅, R₆ and/or R₇) will be null;
   R₁, R₂, R₃, R₄, and R₅ are independently selected from the group consisting of hydrogen, halogen, hydroxy, alkoxy C₁-C₆ linear or branched, alkyl C₁-C₆ linear or branched, wherein at least one of R₁—R₅ is hydroxy, when R₆-R₁₂ are different from hydroxy;
   R₆ and R₇ are independently selected from the group consisting of hydrogen, halogen, hydroxy, C₁-C₆ linear or branched alkoxy, linear or branched C₁-C₆ alkyl, wherein at least one of R₆-R₇ is hydroxy, when R₁-R₅ and R₈-R₁₂ are different from hydroxy; and
   R₈, R₉, R₁₀, R₁₁, and R₁₂ are independently selected from the group consisting of hydrogen, linear or branched C₁-C₆ alkoxy, alkyl linear or branched C₁-C₆; halogen, hydroxy, wherein at least one of R₈-R₁₂ is hydroxy, while R₁-R₇ are different from hydroxy.

The present invention refers to compositions comprising one or -more compound(s) of Formula (I) or a salt, hydrate, solvate and pharmaceutically acceptable isomer thereof; and one or more pharmaceutically acceptable excipients.

In addition, the kits, according to the present invention, may comprise such compositions and application devices, which may include ampoules, syringes and others. Alternatively, the kits according to this invention comprise more than one compound of Formula (I) arranged in one or more dosage forms, including without limitation, tablets, accompanied by administration instructions.

The present invention further refers to methods of treatment, prevention, relief, suppression and/or control of diseases related to neuropathic pain. Uses of Formula (I) compound(s) to prepare a drug for the treatment of pathologies related to neuropathic pain are also taught. Finally, the present invention teaches processes for obtaining compound(s) of Formula (I).

In addition, some compounds described in this invention may exist as tautomers, so the individual tautomers, as well as their mixtures, are included in the compounds with structural formula I.

### DETAILED DESCRIPTION OF THE INVENTION

The present invention presents, in a first embodiment, compounds of Formula (I):

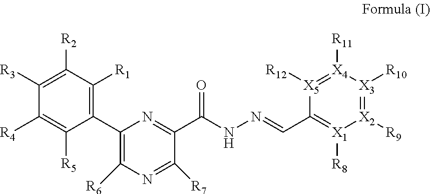

or a pharmaceutically acceptable salt, hydrate, solvate and isomer thereof, wherein:
   one of X₁, X₂, X₃, X₄ and X₅ is nitrogen and the others are carbon, where when one of X₁, X₂, X₃, X₄ and X₅ is nitrogen, the corresponding R (R₅, R₆ and/or R₇) will be null;
R₁, R₂, R₃, R₄, and R₅ are independently selected from the group consisting of hydrogen, halogen, hydroxy, alkoxy C₁-C₆ linear or branched, alkyl C₁-C₆ linear or branched, wherein at least one of R₁-R₅ is hydroxy, when R₆-R₁₂ are different from hydroxy;
R₆ and R₇ are independently selected from the group consisting of hydrogen, halogen, hydroxy, C₁-C₆ linear or branched alkoxy, linear or branched C₁-C₆ alkyl, wherein at least one of R₆-R₇ is hydroxy, when R₁-R₅ and R₈-R₁₂ are different from hydroxy; and
R₈, R₉, R₁₀, R₁₁, and R₁₂ are independently selected from the group consisting of hydrogen, linear or branched C₁-C₆ alkoxy, alkyl linear or branched C₁-C₆, halogen, hydroxy, where at least one of R₈-R₁₂ is hydroxy, while R₁-R₇ are different from hydroxy.

As described herein, the compounds of the present invention comprise multiple variable groups (R, X, etc.). As a person skilled in the matter will recognize, the group combinations contemplated by this invention are those combinations that result in the formation of stable or chemically viable compounds.

The term “stable” in this context refers to compounds that are not substantially altered when subjected to conditions that allow their production, detection and preferably their recovery, purification and use for one or more of the purposes disclosed herein.

In some embodiments, a stable compound or chemically viable compound is one that is not substantially altered when maintained at a temperature of 40° C. or less, in the absence of moisture or other chemically reactive conditions, for at least one week.

In an embodiment, one of X₁, X₂, X₃, X₄ and X₅ is nitrogen and the others are carbon, where when one of X₁, X₂, X₃, X₄ and X₅ is nitrogen, the corresponding R (R₅, R₆, and/or R₇) is null; R₁ is selected from the group consisting of hydrogen, halogen, and hydroxy; R₂ is hydrogen; R₃ is selected from the hydroxy and alkoxy group C₁-C₆ linear or branched; R₄ is selected from the hydrogen and hydroxy group; R₅ is hydrogen; R₆ is selected from the hydrogen and hydroxy group; R₇ is selected from the hydrogen and hydroxy group; R₈ is selected from the hydrogen and hydroxy group, or is null when X₁ is nitrogen; R₉ is selected from the group consisting of hydrogen, hydroxy, linear or branched C₁-C₆ alkoxy, and linear or branched C₁-C₆alkyl; R₁₀ is selected from the group consisting of hydrogen and hydroxy or is null when X₃ is nitrogen; R₁₁ is selected from the group consisting of hydrogen, hydroxy, linear or branched C₁-C₆ alkyl, and C₁-C₆ linear or branched alkoxy, or it is null when X₄ is nitrogen; R₁₂ is selected from the group consisting of hydrogen, hydroxy, and halogen, or it is null when X₅ is nitrogen.

In one embodiment, R₁ is selected from the group consisting of hydrogen, chlorine, bromine, fluorine, and hydroxy. In a preferred embodiment, R₁ is selected from the group consisting of hydrogen, fluorine, and hydroxy.

In one embodiment, R₂ is hydrogen.

In an embodiment, R₃ is selected from the group consisting of hydroxy, methoxy, ethoxy; propoxy, isopropoxy, n-butoxy, sec-butoxy, isobutoxy, tert-butoxy, n-pentoxy, sec-pentoxy, tert-pentoxy, but not limited to same. In a preferred embodiment, R₃ is selected from the group consisting of hydroxy, methoxy, and ethoxy.

In one embodiment, R₄ is selected from the group consisting of hydrogen and hydroxy.

In one embodiment, R₅ is hydrogen.

In one embodiment, R₆ is selected from the group consisting of hydrogen and hydroxy.

In one embodiment, R₇ is selected from the group consisting of hydrogen and hydroxy.

In an embodiment, R₈ is selected from the group consisting of hydrogen and hydroxy or is null when X₁ is nitrogen.

In an embodiment, R₉ is selected from the group consisting of hydrogen, hydroxy, methyl, ethyl, propyl, isopropyl, n-butyl, sec-butyl, tert-butyl, n-pentyl, sec-pentyl, tert-pentyl, methoxy, ethoxy; propoxy, isopropoxy, n-butoxy, sec-butoxy, isobutoxy, tert-butoxy, n-pentoxy, sec-pentoxy, tert-pentoxy, but not limited to same. In a preferred embodiment, R₉ is selected from the group consisting of hydrogen, hydroxy, methyl, and methoxy.

In an embodiment, R₁₀ is selected from the group consisting of hydrogen and hydroxy or is null when X₃ is nitrogen.

In an embodiment, R₁₁ is selected from the group consisting of hydrogen, hydroxy, methyl, ethyl, propyl, isopropyl, n-butyl, sec-butyl, tert-butyl, n-pentyl, sec-pentyl, tert-pentyl, methoxy, ethoxy; propoxy, isopropoxy, n-butoxy, sec-butoxy, iso-butoxy, tert-butoxy, n-pentoxy, sec-pentoxy, tert-pentoxy but not being limited to same, or R₁₁ is null when X₄ is nitrogen. In a preferred embodiment, R₁₁ is selected from the group consisting of hydrogen, hydroxy, methyl, methoxy, or null when X₄ is nitrogen.

In one embodiment, R₁₂ is selected from the group consisting of hydrogen, hydroxy, fluorine, chlorine, bromine, or is null when X₅ is nitrogen. In a preferred embodiment, R₁₂ is selected from the group consisting of hydrogen, hydroxy, fluorine, or is null when X₅ is nitrogen.

In an implementation of the first embodiment, the Formula (I) compound(s) is selected from the group(s) consisting of:
(E)-N′-(3,5-dimethoxybenzylidene)-6-(4-hydroxyphenyl)pyrazine-2-carbohydrazide (Compound 1);
(E)-N′-(3,5-dimethylbenzylidene)-6-(4-hydroxyphenyl)pyrazine-2-carbohydrazide (Compound 2)
(E)-N′-(2-fluoro-5-methoxybenzylidene)-6-(4-hydroxyphenyl)pyrazine-2-carbohydrazide (Compound 3);
(E)-6-(4-hydroxyphenyl)-N′-((5-hydroxypyridine-3-yl)methylene)pyrazine-2-carbohydrazide (Compound 4);
(E)-N′-(3,5-dimethoxybenzylidene)-6-(2-hydroxy-4-methoxyphenyl)pyrazine-2-carbohydrazide (Compound 5);
(E)-N′-(3,5-dimethoxybenzylidene)-6-(3-hydroxy-4-methoxyphenyl)pyrazine-2-carbohydrazide (Compound 6);
(E)-N′-(3,5-dimethylbenzylidene)-6-(2-hydroxy-4-methoxyphenyl)pyrazine-2-carbohydrazide (Compound 7);
(E)-6-(4-ethoxy-2-hydroxyphenyl)-N′-(2-fluoro-5-methoxybenzylidene)pyrazine-2-carbohydrazide (Compound 8);
(E)-6-(4-ethoxy-2-hydroxyphenyl)-N′-((2-fluoro-5-methoxypyridine-3-yl)methylene)pyrazine-2-carbohydrazide (Compound 9);
(E)-6-(4-ethoxy-3-hydroxyphenyl)-N′-((2-fluoro-5-methoxypyridine-3-yl)methylene)pyrazine-2-carbohydrazide (Compound 10);
(E)-6-(4-ethoxy-3-hydroxyphenyl)-N′-(2-fluoro-5-methoxybenzylidene)pyrazine-2-carbohydrazide (Compound 11);
(E)-N′-(3,5-dimethoxybenzylidene)-5-hydroxy-6-(4-methoxyphenyl)pyrazine-2-carbohydrazide (Compound 12);
(E)-N′-(3,5-dimethylbenzylidene)-5-hydroxy-6-(4-methoxyphenyl)pyrazine-2-carbohydrazide (Compound 13);
(E)-N′-(3,5-dimethoxybenzylidene)-3-hydroxy-6-(4-methoxyphenyl)pyrazine-2-carbohydrazide (Compound 14);
(E)-N′-(3,5-dimethylbenzylidene)-3-hydroxy-6-(4-methoxyphenyl)pyrazine-2-carbohydrazide (Compound 15);
(E)-6-(4-ethoxyphenyl)-N′-((2-fluoro-5-methoxypyridine-3-yl)methylene)-5-hydroxypyrazine-2-carbohydrazide (Compound 16);
(E)-6-(4-ethoxyphenyl)-N′-((2-fluoro-5-methoxypyridine-3-yl)methylene)-3-hydroxypyrazine-2-carbohydrazide (Compound 17);
(E)-6-(4-ethoxy-2-fluorophenyl)-5-hydroxy-N′-(3-methoxybenzylidene)pyrazine-2-carbohydrazide (Compound 1.8);
(E)-6-(4-ethoxy-2-fluorophenyl)-3-hydroxy-N′-(3-methoxybenzylidene)pyrazine-2-carbohydrazide (Compound 19);
(E)-N′-(3,5-dihydroxybenzylidene)-6-(4-methoxyphenyl)pyrazine-2-carbohydrazide (Compound 20);
(E)-N′-(3-hydroxy-5-methoxybenzylidene)-6-(4-methoxyphenyl)pyrazine-2-carbohydrazide (Compound 21);
(E)-N′-4-hydroxybenzylidene)-6-(4-methoxyphenyl)pyrazine-2-carbohydrazide (Compound 22);
(E)-N′-(3-hydroxybenzylidene)-6-(4-methoxyphenyl)pyrazine-2-carbohydrazide (Compound 23);
(E)-N′-(2-hydroxybenzylidene)-6-(4-methoxyphenyl)pyrazine-2-carbohydrazide (Compound 24);
(E)-N′-(3-hydroxy-5-methylbenzylidene)-6-(4-methoxyphenyl)pyrazine-2-carbohydrazide (Compound 25);
(E)-6-(4-ethoxyphenyl)-N′-(3-hydroxy-5-methylbenzylidene)pyrazine-2-carbohydrazide (Compound 26);
(E)-6-(4-ethoxyphenyl)-N′-(2-fluoro-5-hydroxybenzylidene)pyrazine-2-carbohydrazide (Compound 27);
(E)-6-(4-ethoxyphenyl)-N′-(2-hydroxy-5-methoxybenzylidene)pyrazine-2-carbohydrazide (Compound 28);
(E)-6-(4-ethoxyphenyl)-N′-((5-hydroxypyridine-3-yl)methylene)pyrazine-2-carbohydrazide (Compound 29);
(E)-6-(4-ethoxyphenyl)-N′-((6-hydroxypyridine-2-yl)methylene)pyrazine-2-carbohydrazide (Compound 30);
(E)-6-(4-ethoxyphenyl)-N′-(2-hydroxy-3,5-dimethoxybenzylidene)pyrazine-2-carbohydrazide (Compound 31);
(E)-6-(4-ethoxyphenyl)-N′-((2-fluoro-5-hydroxypyridine-3-yl)methylene)pyrazine-2-carbohydrazide (Compound 32);
(E)-6-(4-ethoxyphenyl)-N′-((2-fluoro-4-hydroxy-5-methoxypyridine-3-yl)methylene)pyrazine-2-carbohydrazide (Compound 33);
(E)-6-(4-ethoxyphenyl)-N′-((6-hydroxy-4-methoxypyridine-2-yl)methylene)pyrazine-2-carbohydrazide (Compound 34);
(E)-6-(4-ethoxyphenyl)-N′-((2-hydroxy-5-methoxypyridine-yl)methylene)pyrazine-2-carbohydrazide (Compound 35); and
(E)-6-(4-ethoxyphenyl)-N′-((2-hydroxy-6-methoxypyridine-4-yl)methylene)pyrazine-2-carbohydrazide (Compound 36).

In an implementation of the first embodiment, the Formula (I) compound(s) are voltage-gated sodium channel blockers Nav 1.7 and/or Nav 1.8. In a preferred embodiment, the Formula (I) compound(s) are dual voltage-gated sodium channel blockers Nav 1.7 and Nav 1.8.

In an implementation of the first embodiment, the compound(s) of Formula (I) may have a basic nature and, consequently, pharmaceutically acceptable salts may be obtained by the addition of organic or inorganic acids. Non-limiting examples of organic acids that can be used are fumaric, maleic, benzoic, lactic acids, among others. Among the inorganic acids, we can mention hydrochloric acid, phosphoric acid, hydrobromic acid, sulfuric acid, nitric acid, among others.

In an implementation of the first embodiment, the compound(s) of Formula (I) can be obtained in the form of crystals, which can be optionally presented as pharmaceutically acceptable solvates, in which the solvent is incorporated in stoichiometric proportions or not into the crystal lattice. In further embodiments, the crystallization solvent is water, resulting in pharmaceutically acceptable hydrates.

Finally, in an implementation of the first embodiment, the Formula (I) compound(s) may present more than one isomer, including without limitation, spatial isomerism, such as geometric and optical isomerism.

### Definitions

In a first embodiment, the present invention presents phenolic compounds blocking Nav 1.7 and/or Nav 1.8 of Formula (I), or a salt, hydrate, solvate and pharmaceutically acceptable isomer thereof.

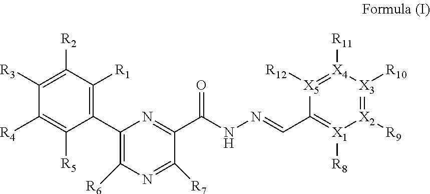

In order to clarify or elucidate the terms used in this invention, the following definitions are presented, whereby the scope is not limited thereto.

The term “halogen” refers to the elements of the 7A family of the periodic table, which are: fluorine (F), chlorine (Cl), bromine (Br), iodine (I), astatine (At) and tennessine (Ts).

The term “linear or branched C₁-C₆ alkyl refers to saturated straight- or branched-chain hydrocarbons such as for example, methyl, ethyl, propyl, isopropyl, n-butyl, sec-butyl, tert-butyl, n-pentyl, n-pentyl, sec-pentyl, tert-pentyl, but not limited to them.

The term “linear or branched C₁-C₆ alkoxy” refers to alkyl groups attached to an oxygen radical, such as methoxy, ethoxy; propoxy, isopropoxy, n-butoxy, sec-butoxy, iso-butoxy, tert-butoxy, n-pentoxy, sec-pentoxy, tert-pentoxy, but not limited to same.

In a second embodiment, the present invention presents a composition comprising a therapeutically effective amount of compound(s) of Formula (I) of the present invention or a pharmaceutically acceptable salt, hydrate, solvate, and isomer thereof; and one or more pharmaceutically acceptable excipients.

Pharmaceutically acceptable excipients are any substance, other than the active pharmaceutical ingredient, that has been evaluated for its safety and that is intentionally added to the dosage form. Such excipients are selected according to the pharmaceutical dosage form of interest, their route of administration, physicochemical compatibility with the active ingredient, and the effect on efficacy.

In addition, these excipients are widely known in the statie of the art and are classified according to their function, including without limitation diluents, binders, disintegrants or disaggregator, lubricants, suspending agents, thickeners, solvents, surfactants, sliders, anti-caking agents or flow agents, glazing agents, plasticizers, sweeteners, isotonicity agents, dyes and pigments, preservatives, antioxidants, modifying agents or pH control, complexing agents, chelating agents, flavorings, viscosity modifying agents, opacifiers, permeation promoters, among others.

In an implementation of the second embodiment, the pharmaceutical compositions of the present invention can be administered by several routes including oral, sublingual, nasal, parenteral, injectable, submuscular, topical, transdermal, ocular, rectal, but not limited to these.

In a third embodiment, the present invention presents the use of compound(s) of Formula (I) or a salt, hydrate, solvate and pharmaceutically acceptable isomer thereof to prepare a drug to treat pathologies related to neuropathic pain.

In an implementation of the third embodiment, said pathologies are selected from the group consisting of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

In a fourth embodiment, the present invention presents a method of treatment, prevention, relief, suppression and/or control of pathologies related to neuropathic pain comprising the administration of an effective amount of Formula (I) compound(s) or a salt, hydrate, solvate and pharmaceutically acceptable isomer thereof.

In an implementation of the fourth embodiment, the method is for the treatment, prevention, relief, suppression and/or control of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

In another implementation of the fourth embodiment, the administration of at least one Formula (I) compound is selected from the group comprising oral, sublingual, nasal, parenteral, injectable, submuscular, topical, transdermal, ocular and rectal routes.

In a fifth embodiment, the present invention presents processes for obtaining compound(s) of Formula (I) comprising the following steps:
(a) Formation of the Formula III intermediate:

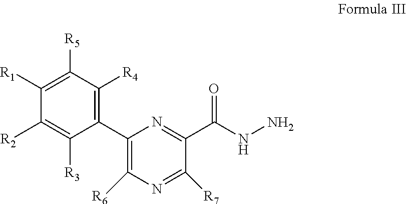

from the hydrazinolysis reaction of a Formula IV intermediate:

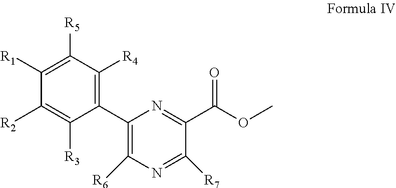

(b) obtaining Formula I compound;

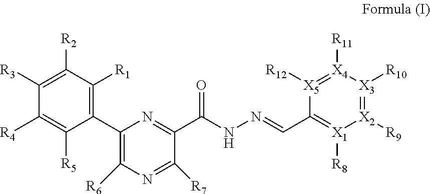

from the condensation of Formula II intermediates:

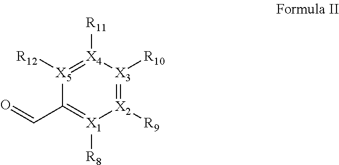

and Formula III with or without the presence of a catalyst end a suitable solvent;
wherein,
   one of X₁, X₂, X₃, X₄ and X₅ is nitrogen and the others are carbon, wherein when one of X₁, X₂, X₃, X₄ and X₅ is nitrogen, the corresponding R (R₅, R₆ and/or R₇) will be null;
   R₁, R₂, R₃, R₄, and R₅ are independently selected from the group consisting of hydrogen, halogen, hydroxy, alkoxy C₁-C₆ linear or branched, alkyl C₁-C₆ linear or branched alkoxy, wherein at least one of R₁-R₅ is hydroxy, when R₆-R₁₂ are different from hydroxy;
   R₆ and R₇ are independently selected from hydrogen, halogen, hydroxy, C₁-C₆ linear or branched alkyl, C₁-C₆ linear or branched alkoxy, wherein at least one of R₆₋₇ is hydroxy, when R₁-R₅ and R₆-R₁₂ are different from hydroxy; and
   R₈, R₉, R₁₀, R₁₁, and R₁₂ are independently selected from the group consisting of hydrogen, linear or branched C₁-C₆ alkoxy, alkyl linear or branched C₁-C₆, halogen, hydroxy, where at least one of R₆-R₁₂ is hydroxy, while R₁-R₇ are different from hydroxy.

In an implementation of the fifth embodiment, said catalyst of step (b) is selected from concentrated hydrochloric acid, acetic acid, trifluoroacetic acid, formic acid, or combinations thereof, and the solvent is selected from dimethylformamide, alcohols, or combinations thereof.

The compound(s) of Formula (I) of this invention have been prepared from the synthetic route described in General Scheme 1. However, those skilled in the art will readily notice that, additional detailing and/or modifications to the arrangement of one or more steps can be accomplished without departing from the processes taught herein. Such variations can be, without limitation, combinations of solvents and catalysts, including stereoselective ones, protective groups, among others.

In the following General Scheme 1, the formation of the Formula II, III and IV intermediates is described, in addition to the formation of the Formula (I) compound(s).

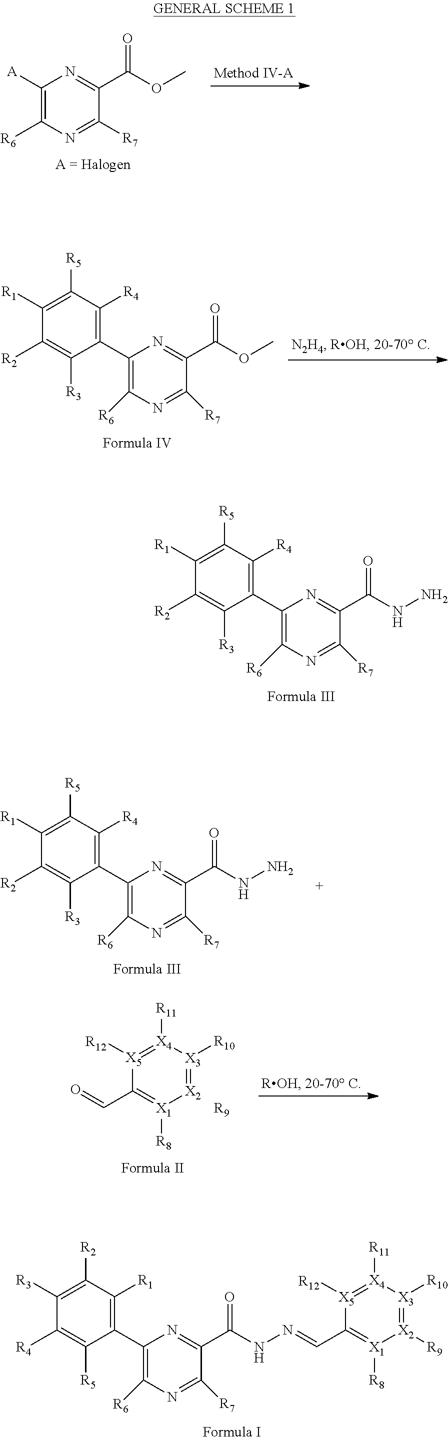

### Formula IV Intermediates

Following the General Scheme 1, the method for the formation of the formula IV intermediates is presented, however such methods are not limiting.

### Method IV-A

### Intermediates IV-1 to IV-8

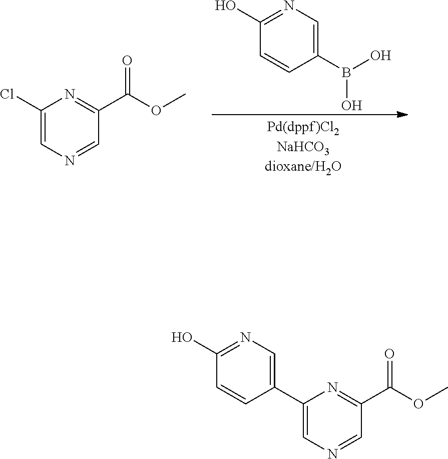 [SMILES: COC(=O)c1cncc(Cl)n1 ; OB(O)c1ccc(O)nc1 ; COC(=O)c1cncc(-c2ccc(O)nc2)n1]

In a round-bottomed flask containing methyl 6-chloropyrazine-2-carboxylate (11.6 mmol) in dioxane (50.0 mL) and H₂O (10.0 mL), (6-hydroxypyridine-3-yl) boronic acid (12.8 mmol), NaHCO₃ (17.4 mmol), and Pd(dppf)Cl₂ (579 μmol) were added under N₂ atmosphere. The reaction mixture was kept under agitation at 70° C. for 12 hours. After full consumption of the starting reagent by (monitored CCD), the mixture was concentrated under reduced pressure to remove the solvent. The residue obtained was diluted with H₂O (100 mL) and extracted with AcOEt (100 mL×3). The organic phase was concentrated under reduced pressure and purified by column chromatography (petroleum ether/AcOEt: 50/1→0/1). 1.5 g (56% yield) of the corresponding ester were obtained in the form of a white solid.

TABLE 1
INTERMEDIATES OBTAINED FROM THE CORRESPONDING
REACTANTS FOLLOWING THE METHOD
DESCRIBED IN OBTAINING FORMULA IV INTERMEDIATES.

| Intermediate | Structure |
|---|---|
| IV-1 | 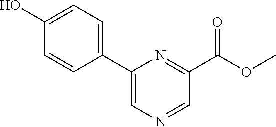 [SMILES: COC(=O)c1cncc(-c2ccc(O)cc2)n1] |
| IV-2 | 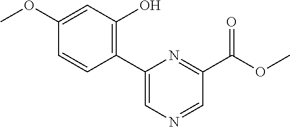 |
| IV-3 | 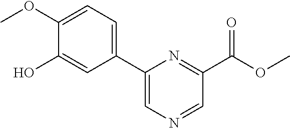 |
| IV-4 | 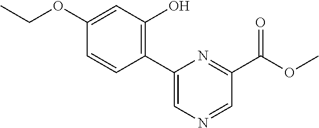 [SMILES: CCOc1ccc(-c2cncc(C(=O)OC)n2)c(O)c1] |
| IV-5 | 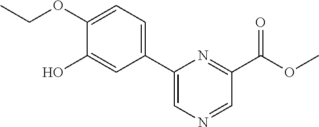 [SMILES: CCOc1ccc(-c2cncc(C(=O)OC)n2)cc1O] |
| IV-6 | 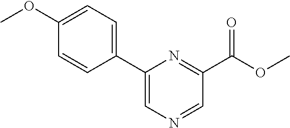 |
| IV-7 | 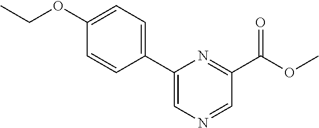 [SMILES: CCOc1ccc(-c2cncc(C(=O)OC)n2)cc1] |
| IV-8 | 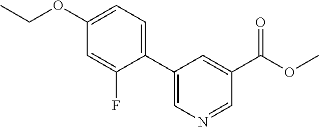 [SMILES: CCOc1ccc(-c2cncc(C(=O)OC)c2)c(F)c1] |

### Obtaining the Formula III Intermediates

Following General Scheme 1, the formula III intermediates are presented.

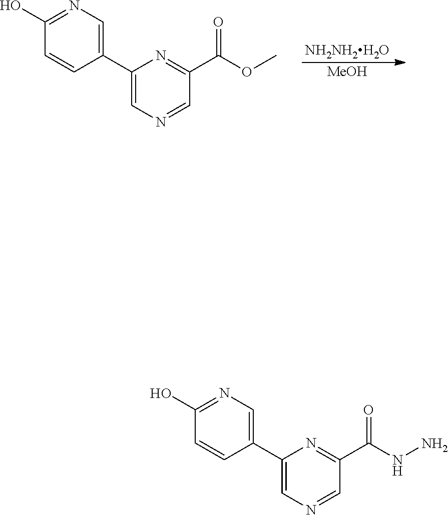

In a round-bottomed flask containing methyl 6-(6-hydroxypyridine-3-yl)pyrazine-2-carboxylate (11.6 mmol) in MeOH (30.0 mL), N₂H₄·H₂O (17.4 mmol) was added. The reaction mixture was kept under agitation at 60° C. for 3 hours. After total consumption of the starting reagent (monitored by CCD), the mixture was filtered and concentrated. 1.6 g (60.0% yield) of the hydrazide of interest were obtained in the form of a white solid, which was sent to the next step without further purification (Table 2).

TABLE 2
INTERMEDIATES OBTAINED FROM THE CORRESPONDING
REAGENTS FOLLOWING THE METHOD DESCRIBED IN
OBTAINING THE COMPOUNDS OF FORMULA III.

| Intermediate | Structure |
|---|---|
| III-1 | 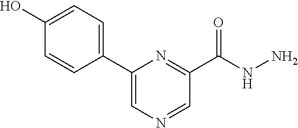 [SMILES: NNC(=O)c1cncc(-c2ccc(O)cc2)n1] |
| III-2 | 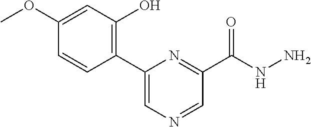 [SMILES: COc1ccc(-c2cncc(C(=O)NN)n2)c(O)c1] |
| III-3 | 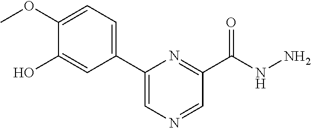 [SMILES: COc1ccc(-c2cncc(C(=O)NN)n2)cc1O] |
| III-4 | 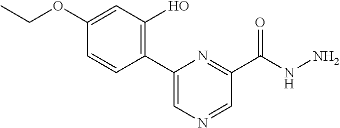 [SMILES: CCOc1ccc(-c2cncc(C(=O)NN)n2)c(O)c1] |
| III-5 | 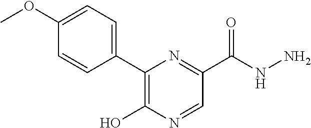 [SMILES: COc1ccc(-c2nc(C(=O)NN)cnc2O)cc1] |
| III-6 | 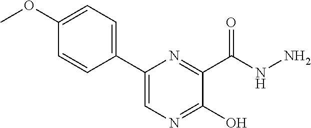 [SMILES: COc1ccc(-c2cnc(O)c(C(=O)NN)n2)cc1] |
| III-7 | 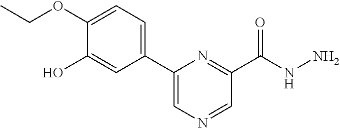 [SMILES: CCOc1ccc(-c2cncc(C(=O)NN)n2)cc1O] |
| III-8 | 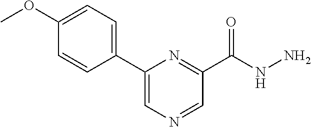 [SMILES: COc1ccc(-c2cncc(C(=O)NN)n2)cc1] |
| III-9 | 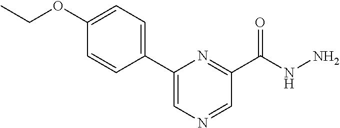 [SMILES: CCOc1ccc(-c2cncc(C(=O)NN)n2)cc1] |
| III-10 | 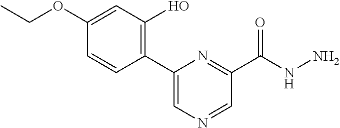 [SMILES: CCOc1ccc(-c2cncc(C(=O)NN)n2)c(O)c1] |
| III-11 | 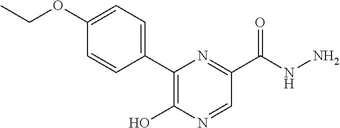 [SMILES: CCOc1ccc(-c2nc(C(=O)NN)cnc2O)cc1] |
| III-12 | 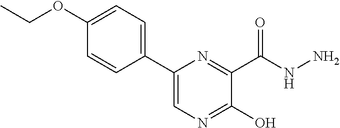 [SMILES: CCOc1ccc(-c2cnc(O)c(C(=O)NN)n2)cc1] |
| III-13 | 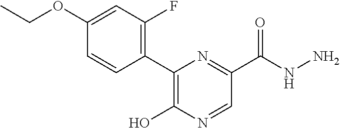 [SMILES: CCOc1ccc(-c2nc(C(=O)NN)cnc2O)c(F)c1] |
| III-14 | 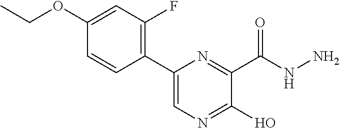 [SMILES: CCOc1ccc(-c2cnc(O)c(C(=O)NN)n2)c(F)c1] |

### Obtaining Formula II Compounds

Following General Scheme 1, there are now presented methods for the formation of Formula II intermediates without limitations.

### Method II-A

### Step II-A-I:

### Precursor II-1I:

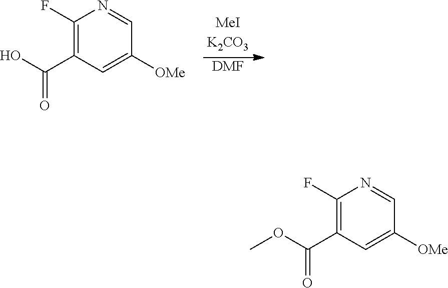

In a round-bottomed flask containing 2-fluoro-5-methoxynicotinic acid (64.3 mmol) and K₂CO₃ (96.4 mmol) in DMF (50.0 mL), iodomethane (129 mmol) were added. Subsequently, the reaction mixture was kept in agitation at 30° C. for 16 hours. After total consumption of the starting reagent (monitored by LC-MS), the reaction mixture was diluted with H₂O (200 mL) and extracted with AcOEt (50.0 mL×3). The organic phase was washed with saturated sodium chloride solution (brine) (100 mL×2), dried on anhydrous sodium sulfate, filtered, concentrated under reduced pressure. 10.0 g (84.0% yield) of the product of interest was obtained in the form of a brown solid, which was used in the next step without further purification.

### Step II-A-II:

### Precursor II-1II:

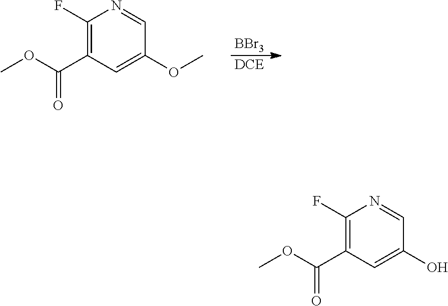

In a round-bottomed flask containing methyl 2-fluoro-5-methoxynicotinate (91.8 mmol) in DCE (400 mL), BBr₃ (230 mmol) was added at 0° C. Subsequently, the reaction mixture was kept in agitation at 80° C. for 4 hours. After total consumption of the starting reagent (monitored by LC-MS), the reaction mixture was cooled to 20° C., diluted with H₂O (200 mL) and extracted with DCM/MeOH: 5/1 (100 mL×5). The organic phase was washed with saturated sodium chloride solution (brine) (100 mL), dried on anhydrous sodium sulfate, filtered and concentrated under reduced pressure. 11.0 g (70.0% yield) of the product of interest were obtained in the form of a brown solid, which was used for the next step without further purification.

### Step II-A-II:

### Precursor II-1II:

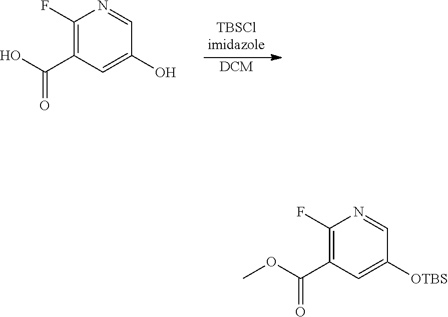

TBSCl (45.0 mmol) at 0° C. was added to a round-bottomed flask containing 2-fluoro-5-hydroxynicotinic acid. (40.9 mmol) and imidazole (81.8 mmol) in DCM (50.0 mL). Subsequently, the reaction mixture was kept in agitation at 20° C. for 16 hours. After total consumption of the starting reagent (monitored by CCD), the reaction mixture was diluted with H₂O (50.0 mL) and extracted with DCM (50.0 mL×3). Subsequently, the organic phase was concentrated under reduced pressure and purified by column chromatography (petroleum ether/AcOEt: 50/1→0/1). 6.50 g (55.7% yield) of the product of interest were obtained in the form of a colorless oil.

### Step II-A-III:

### Precursor III-1III:

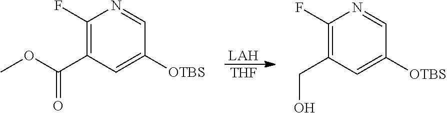 [SMILES: COC(=O)c1cc(O[Si](C)(C)C(C)(C)C)cnc1F ; "LAH" ; CC(C)(C)[Si](C)(C)Oc1cnc(F)c(CO)c1 ; "THF"]

In a round-bottomed flask containing methyl 5-((tert-butyldimethylsilyl)oxy)-2-fluoronicotinate (22.8 mmol) in THF (50.0 mL), LiAlH₄ (45.6 mmol) was added at 0° C. Subsequently, the reaction mixture was kept in agitation at 20° C. for 1 hour. After full consumption of the starting reagent (monitored by CCD), Na₂SO₄·10H was added to the reaction mixture ₂O (3.00 g). Next, the mixture was filtered and extracted with AcOEt (10.0 mL×3). The organic phase was concentrated under reduced pressure. 4.00 g (68.2% yield) of the product of interest were obtained in the form of a yellow oil, which was used for the next step without further purification.

### Step II-A-III:

### Precursor II-1III:

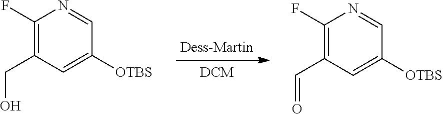 [SMILES: CC(C)(C)[Si](C)(C)Oc1cnc(F)c(CO)c1 ; CC(C)(C)[Si](C)(C)Oc1cnc(F)c(C=O)c1]

In a round-bottomed flask containing (5-((tert-butyldimethylsilyl)oxy)-2-fluoropyridin-3-yl)methanol (15.5 mmol) in DCM (30.0 mL), 1,1-diacetoxy-3-oxo-1,2-benziodoxol-1-yla acetate (23.3 mmol) was added at 0° C. Subsequently, the reaction mixture was kept in agitation at 20° C. for 16 hours. After total consumption of the starting reagent (monitored by LC-MS and CCD), a solution of NaHCO₃ was added to the reaction mixture up to pH=8. Next, the mixture was filtered, diluted with H₂O (50.0 mL) and extracted with DCM (50.0 mL×3). The organic phase was concentrated under reduced pressure and purified by column chromatography (petroleum ether/AcOEt: 10/1→0/1). 3.20 g (80.6% yield) of the product of interest were obtained in the form of a yellow oil.

### Step II-A-IV:

### Intermediate II-1

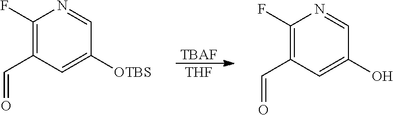 [SMILES: CC(C)(C)[Si](C)(C)Oc1cnc(F)c(C=O)c1 ; "TBAF" ; O=Cc1cc(O)cnc1F ; "THF"]

In a round-bottomed flask containing 5-((tert-butyldimethylsilyl)oxy)-2-fluoronicotinaldehyde (12.5 mmol) in THF (15.0 mL), TBAF (1.00 M, 18.8 mL) were added. Subsequently, the reaction mixture was kept in agitation at 20° C. for 1 hour. After total consumption of the starting reagent (monitored by LC-MS and CCD), the reaction mixture was diluted with H₂O (10.0 mL) and extracted with AcOEt (10.0 mL×3). Subsequently, the organic phase was washed with saturated sodium chloride solution (brine) (10.0 mL), dried on anhydrous sodium sulfate, filtered and concentrated under reduced pressure. The resulting product was purified by column chromatography (petroleum ether/AcGEt: 10/1-0/1). 1.50 g (84.8% yield) of the product of interest were obtained in the form of a white solid.

### Method II-B

### Step II-B-I:

### Intermediate II-2

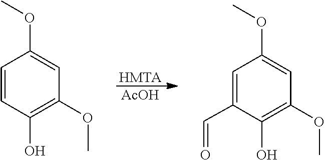

In a round-bottomed flask containing 2,4-dimethoxyphenol (25.95 mmol) in AcOH (40 mL), HMTA (51.89 mmol) were added. Subsequently, the reaction mixture was kept in agitation at 95° C. for 6 hours. After total consumption of the starting reagent (monitored by CCD), the reaction mixture was diluted with H₂O (100 mL), an HCl solution was added (1N, 10 mL), and the mixture was left under agitation for 1 hour. Subsequently, the mixture was extracted with AcOEt (300 mL) and the organic phase was dried on anhydrous sodium sulfate, filtered and concentrated under reduced pressure. The resulting product was purified by column chromatography (petroleum ether/AcOEt: 30/1→10/1). 456 mg (9.65% yield) of the product of interest were obtained in the form of a yellow solid.

### EXAMPLES

The following examples, described in detail, serve to illustrate the embodiments of this invention without, however, having any limitation character to the scope of protection thereof.

The General Formula I compound(s) of this invention were obtained from the condensation of Formula III intermediates with commercial aldehydes or with the Formula II intermediates described above, synthesized according to the various methodologies previously described and schematized in General Scheme 1, but not limited to these.

Compounds 1 to 36, as shown in Table 3, are obtained by the procedure described for Example 1 (Compound 1), changing the corresponding intermediates II and III.

### Example 1. (E)-N′-(3,5-Dimethoxybenzylidene)-6-(6-Hydroxypyridine-3-IL)Pyrazine-2-Carbohydrazide

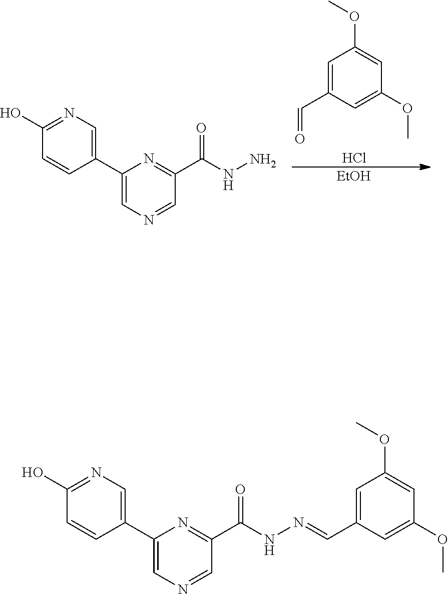 [SMILES: "EtOH" ; NNC(=O)c1cncc(-c2ccc(O)nc2)n1 ; COc1cc(C=O)cc(OC)c1 ; Cl ; COc1cc(/C=N/NC(=O)c2cncc(-c3ccc(O)nc3)n2)cc(OC)c1]

In a round-bottomed flask, 6-(6-hydroxypyridine-3-yl)pyrazine-2-carbohydrazide (2.08 mmol) were added to an aqueous solution of HCl (20.81 mmol). The reaction mixture was kept under agitation for 16 hours. After the reaction was finished, the mixture was concentrated under reduced pressure. The solid obtained was washed with hexane (20 mL×3) and H₂O (20 mL×3), to obtain 540 mg (64% yield) of the product of interest 1 in the form of a white solid.

TABLE 3
COMPOUNDS OBTAINED FROM THE CORRESPONDING REACTANTS FOLLOWING THE I-A METHOD.

| Compound | | ¹H-NMR/ |
|---|---|---|
| N^{o} | Structure | MS (m/z) [M + H]⁺ |
| 1 | 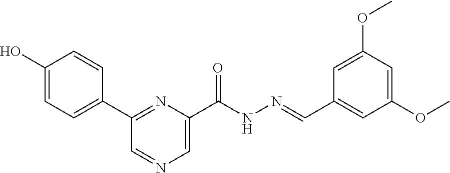 [SMILES: COc1cc(/C=N/NC(=O)c2cncc(-c3ccc(O)cc3)n2)cc(OC)c1] | ¹H-NMR, 400 MHz, DMSO-d₆ δ = 12.03 (br s, 1H), 10.18-9.87 (m, 1H), 9.41 (s, 1H), 9.07 (s, 1H), 8.66 (s, 1H), 8.31 (d, J = 8.8 Hz, 2H), 7.04-6.89 (m, 4H), 6.62 (t, J = 2.3 Hz, 1H), 3.82 (s, 6H). [M + H]⁺: 379.1. |
| | (E)-N′-(3,5- | |
| | dimethoxybenzylidene)-6- | |
| | (4- | |
| | hydroxyphenyl)pyrazine- | |
| | 2-carbohydrazide | |
| 2 | 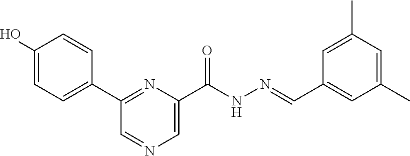 [SMILES: Cc1cc(C)cc(/C=N/NC(=O)c2cncc(-c3ccc(O)cc3)n2)c1] | ¹H-NMR, 400 MHz, DMSO-d₆ δ = 11.99 (br s, 1H), 10.05 (br s, 1H), 9.41 (s, 1H), 9.07 (s, 1H), 8.64 (s, 1H), 8.31 (br d, J = 8.7 Hz, 2H), 7.40 (s, 2H), 7.12 (s, 1H), 6.96 (br d, J = 8.7 Hz, 2H), 2.35 (s, 6H). [M + H]⁺: 347.1. |
| | (E)-N′-(3,5- | |
| | dimethylbenzylidene)-6- | |
| | (4- | |
| | hydroxyphenyl)pyrazine- | |
| | 2-carbohydrazide | |
| 3 | 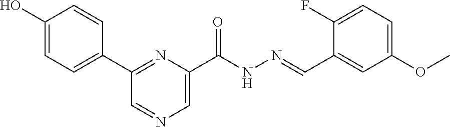 [SMILES: COc1ccc(F)c(/C=N/NC(=O)c2cncc(-c3ccc(O)cc3)n2)c1] | ¹H-NMR, 400 MHz, DMSO-d₆ δ = 12.21 (s, 1H), 10.05 (br s, 1H), 9.42 (s, 1H), 9.08 (s, 1H), 8.98 (s, 1H), 8.32 (d, J = 8.7 Hz, 2H), 7.44 (dd, J = 3.2, 5.5 Hz, 1H), 7.29 (t, J = 9.5 Hz, 1H), 7.10 (td, J = 3.8, 8.9 Hz, 1H), 6.97 (d, J = 8.8 Hz, 2H), 3.83 (s, 3H). [M + H]⁺: 367.1. |
| | (E)-N′-(2-fluoro-5- | |
| | methoxybenzylidene)-6- | |
| | (4- | |
| | hydroxyphenyl)pyrazine- | |
| | 2-carbohydrazide | |
| 4 | 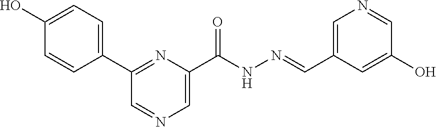 [SMILES: O=C(N/N=C/c1cncc(O)c1)c1cncc(-c2ccc(O)cc2)n1] | ¹H-NMR, 400 MHz, DMSO-d₆ δ = 6.96 (d, J = 8.76 Hz, 2 H) 7.57-7.62 (m, 1 H) 8.21 (d, J = 2.75 Hz, 1 H) 8.27-8.35 (m, 3 H) 8.72 (s, 1 H) 9.07 (s, 1 H) 9.41 (s, 1 H) 9.94-10.33 (m, 2 H) 12.14 (s, 1 H). [M + H]⁺: 336.4. |
| | (E)-6-(4-hydroxyphenyl)- | |
| | N′-((5-hydroxypyridine- | |
| | 3-yl)methylene)pyrazine- | |
| | 2-carbohydrazide | |
| 5 | 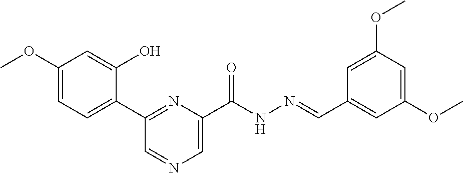 | ¹H-NMR, 400 MHz, DMSO-d₆ δ = 12.06 (s, 1 H), 10.84 (s, 1H), 9.51 (s, 1H), 9.04 (s, 1H), 8.60 (s, 1H), 8.24 (d, J = 8.7 Hz, 1H), 6.93 (d, J = 2.1 Hz, 2H), 6.71-6.53 (m, 3H), 3.82 (d, J = 1.6 Hz, 9H). [M + H]⁺: 409.1. |
| | (E)-N′-(3,5- | |
| | dimethoxybenzylidene)-6- | |
| | (2-hydroxy-4- | |
| | methoxyphenyl)pyrazine- | |
| | 2-carbohydrazide | |
| 6 | 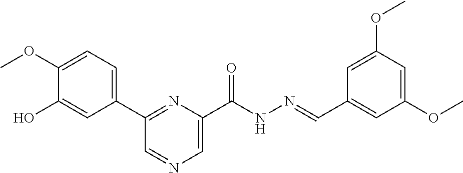 | ¹H-NMR, 400 MHz, DMSO-d₆ δ = 12.10 (br s, 1H), 9.38 (s, 1H), 9.23 (br s, 1H), 9.08 (s, 1H), 8.64 (s, 1H), 7.91-7.81 (m, 2H), 7.11 (d, J = 8.8 Hz, 1H), 6.93 (d, J = 2.2 Hz, 2H), 6.62 (t, J = 2.1 Hz, 1H), 3.89 (s, 3H), 3.82 (s, 6 H). [M + H]⁺: 409.1. |
| | (E)-N′-(3,5- | |
| | dimethoxybenzylidene)-6- | |
| | (3-hydroxy-4- | |
| | methoxyphenyl)pyrazine- | |
| | 2-carbohydrazide | |
| 7 | 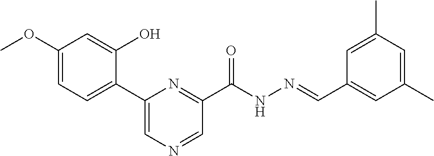 | ¹H-NMR, 400 MHz, DMSO-d₆ δ = 12.02 (s, 1H), 10.85 (s, 1H), 9.50 (s, 1H), 9.04 (s, 1H), 8.58 (s, 1H), 8.22 (br d, J = 8.7 Hz, 1H), 7.39 (s, 2H), 7.17-7.08 (m, 1H), 6.72-6.55 (m, 2H), 3.81 (s, 3H), 2.34 (s, 6H). [M + H]⁺: 377.1. |
| | (E)-N′-(3,5- | |
| | dimethylbenzylidene)-6- | |
| | (2-hydroxy-4- | |
| | methoxyphenyl)pyrazine- | |
| | 2-carbohydrazide | |
| 8 | 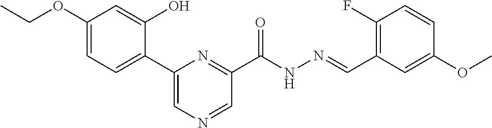 [SMILES: CCOc1ccc(-c2cncc(C(=O)N/N=C/c3cc(OC)ccc3F)n2)c(O)c1] | ¹H-NMR, 400 MHz, DMSO-d₆ δ = 12.23 (s, 1H), 10.78 (s, 1H), 9.52 (s, 1H), 9.04 (s, 1H), 8.92 (s, 1H), 8.23 (d, J = 8.8 Hz, 1H), 7.43 (dd, J = 3.3, 5.6 Hz, 1H), 7.28 (t, J = 9.6 Hz, 1H), 7.09 (td, J = 3.8, 8.8 Hz, 1H), 6.69-6.54 (m, 2H), 4.08 (q, J = 7.0 Hz, 2H), 3.83 (s, 3H), 1.36 (t, J = 6.9 Hz, 3H). [M + H]⁺: 411.4. |
| | (E)-6-(4-ethoxy-2- | |
| | hydroxyphenyl)-N′-(2- | |
| | fluoro-5- | |
| | methoxybenzylidene)pyrazine- | |
| | 2-carbohydrazide | |
| 9 | 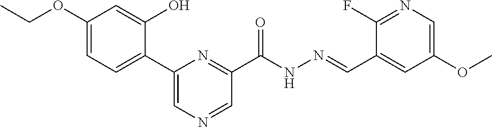 [SMILES: CCOc1ccc(-c2cncc(C(=O)N/N=C/c3cc(OC)cnc3F)n2)c(O)c1] | ¹H-NMR, 400 MHz, DMSO-d₆ δ = 12.35 (br s, 1H), 10.85-10.64 (m, 1H), 9.54-9.49 (m 1H), 9.04 (s, 1H), 8.86-8.82 (m, 1H), 8.21 (d, J = 8.8 Hz, 1H), 8.02 (dd, J = 1.8, 3.0 Hz, 1H), 7.90 (dd, J = 3.1, 7.6 Hz, 1H), 6.67-6.61 (m, 1H), 6.57 (d, J = 2.4 Hz, 1H), 4.07 (q, J = 7.0 Hz, 2H), 3.91 (s, 3H), 1.36 (t, J = 6.9 Hz, 3H). [M + H]⁺: 412.0. |
| | (E)-6-(4-ethoxy-2- | |
| | hydroxyphenyl)-N′-((2- | |
| | fluoro-5- | |
| | methoxypyridine-3- | |
| | yl)methylene)pyrazine-2- | |
| | carbohydrazide | |
| 10 | 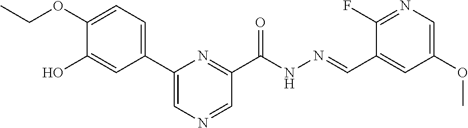 [SMILES: CCOc1ccc(-c2cncc(C(=O)N/N=C/c3cc(OC)cnc3F)n2)cc1O] | |
| | (E)-6-(4-ethoxy-3-hydroxyphenyl)-N′-((2-fluoro-5- | |
| | methoxypyridine-3-yl)methylene)pyrazine-2- | |
| | carbohydrazide | |
| 11 | 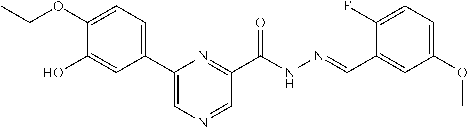 [SMILES: CCOc1ccc(-c2cncc(C(=O)N/N=C/c3cc(OC)ccc3F)n2)cc1O] | |
| | (E)-6-(4-ethoxy-3-hydroxyphenyl)-N′-(2-fluoro-5- | |
| | methoxybenzylidene)pyrazine-2-carbohydrazide | |
| 12 | 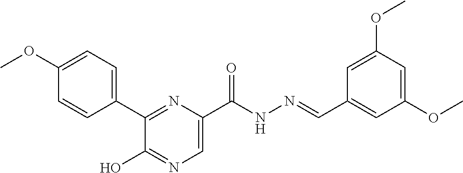 | ¹H-NMR, 400 MHz, DMSO-d₆ δ = 13.07 (s, 1H), 11.48 (br d, J = 0.7 Hz, 1H), 8.63-8.44 (m, 3H), 7.99 (s, 1H), 7.01 (br d, J = 9.0 Hz, 2H), 6.87 (br d, J = 1.8 Hz, 2H), 6.59-6.53 (m, 1H), 3.82 (s, 3H), 3.78 (s, 6H). [M + H]⁺: 409.1. |
| | (E)-N′-(3.5- | |
| | dimethoxybenzylidene)-5- | |
| | hydroxy-6-(4- | |
| | methoxyphenyl)pyrazine- | |
| | 2-carbohydrazide | |
| 13 | 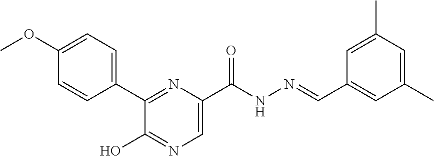 | ¹H-NMR, 400 MHz, DMSO-d₆ δ = 13.02-12.75 (m, 1H), 11.46 (s, 1H), 8.66-8.51 (m, 3H), 8.02 (s, 1H), 7.36 (s, 2H), 7.13-7.01 (m, 3H), 3.85 (s, 3H), 2.33 (s, 6H). [M + H]⁺: 377.1. |
| | (E)-N′-(3.5- | |
| | dimethybenzylidene)-5- | |
| | hydroxy-6-(4- | |
| | methoxyphenyl)pyrazine- | |
| | 2-carbohydrazide | |
| 14 | 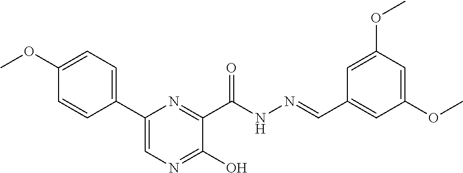 | ¹H-NMR, 400 MHz, DMSO-d₆ δ = 8.48 (br d, J = 6.7 Hz, 2H), 8.08-7.61 (m, 2H), 7.10-6.89 (m, 4H), 6.73-6.31 (m, 2H), 3.86-3.74 (m, 9H). [M + H]⁺: 409.1. |
| | (E)-N′-(3.5- | |
| | dimethoxybenzylidene)-3- | |
| | hydroxy-6-(4- | |
| | methoxyphenyl)pyrazine- | |
| | 2-carbohydrazide | |
| 15 | 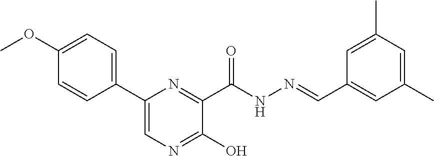 | ¹H-NMR, 400 MHz, DMSO-d₆ δ = 8.58-8.22 (m, 2H), 7.94 (br d, J = 7.3 Hz, 2H), 7.39 (s, 2H), 7.18-6.87 (m, 3H), 3.83-3.78 (m, 3H), 2.34 (s, 5H). [M + H]⁺: 377.1. |
| | (E)-N′-(3.5- | |
| | dimethylbenzylidene)-3- | |
| | hydroxy-6-(4- | |
| | methoxyphenyl)pyrazine- | |
| | 2-carbohydrazide | |
| 16 | 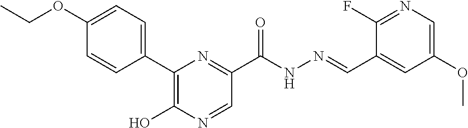 [SMILES: CCOc1ccc(-c2nc(C(=O)N/N=C/c3cc(OC)cnc3F)cnc2O)cc1] | ¹H-NMR, 400 MHz, DMSO-d₆ δ = 13.05-12.74 (m, 1H), 11.85 (s, 1H), 8.82 (s, 1H), 8.63 (d, J = 1.4 Hz, 2H), 8.05-7.98 (m, 2H), 7.87 (dd, J = 3.0, 7.8 Hz, 1H), 7.03 (d, J = 9.0 Hz, 2H), 4.12 (d, J = 7.0 Hz, 2H), 3.91 (s, 3H), 1.37 (t, J = 7.0 Hz, 3H). [M + H]⁺: 412.0. |
| | (E)-6-(4-ethoxyphenyl)- | |
| | N′-((2-fluoro-5- | |
| | methoxypyridine-3- | |
| | yl)methylene)-5- | |
| | hydroxypyrazine-2- | |
| | carbohydrazide | |
| 17 | 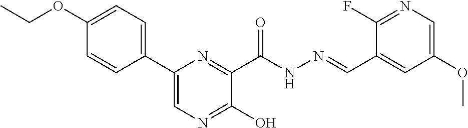 [SMILES: CCOc1ccc(-c2cnc(O)c(C(=O)N/N=C/c3cc(OC)cnc3F)n2)cc1] | |
| | (E)-6-(4-ethoxyphenyl)-N′-((2-fluoro-5-methoxypyridine- | |
| | 3-yl)methylene)-3-hydroxypyrazine-2-carbohydrazide | |
| 18 | 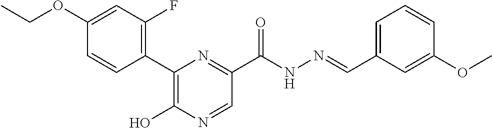 [SMILES: CCOc1ccc(-c2nc(C(=O)N/N=C/c3cccc(OC)c3)cnc2O)c(F)c1] | ¹H-NMR, 400 MHz, DMSO-d₆ δ = 12.96 (td, J = 1.5, 2.8 Hz, 1H), 11.55-10.73 (m, 1H), 8.51 (s, 1H), 8.09 (br s, 1H), 7.65 (br t, J = 8.5 Hz, 1H), 7.43-7.33 (m, 1H), 7.29-7.20 (m, 2H), 7.01 (br d, J = 8.8 Hz, 1H), 6.94-6.86 (m, 2H), 4.12 (q, J = 6.9 Hz, 2H), 3.82-3.76 (m, 3H), 1.36 (br t, J = 6.8 Hz, 3H). [M + H]⁺: 411.1. |
| | (E)-6-(4-ethoxy-2- | |
| | fluorophenyl)-5-hydroxy- | |
| | N′-(3- | |
| | methoxybenzylidene)pyrazine- | |
| | 2-carbohydrazide | |
| 19 | 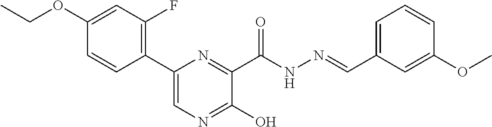 [SMILES: CCOc1ccc(-c2cnc(O)c(C(=O)N/N=C/c3cccc(OC)c3)n2)c(F)c1] | ¹H-NMR, 400 MHz, DMSO-d₆ δ = 12.44-12.09 (m, 1H), 8.44 (s, 1H), 8.00-7.88 (m, 1H), 7.72 (t, J = 9.2 Hz, 1H), 7.52-7.21 (m, 3H), 7.11-6.81 (m, 4H), 4.09 (quin, J = 6.8 Hz, 2H), 3.83-3.66 (m, 3H), 1.38-1.30 (m, 3H). [M + H]⁺: 411.2. |
| | (E)-6-(4-ethoxy-2- | |
| | fluorophenyl)-3-hydroxy- | |
| | N′-(3- | |
| | methoxybenzylidene)pyrazine- | |
| | 2-carbohydrazide | |
| 20 | 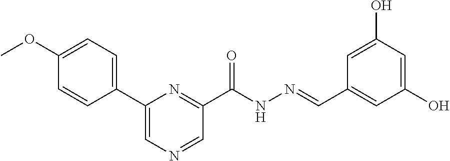 [SMILES: COc1ccc(-c2cncc(C(=O)N/N=C/c3cc(O)cc(O)c3)n2)cc1] | ¹H-NMR, 400 MHz, DMSO-d₆ δ = 11.95 (s, 1H), 9.49 (d, J = 15.9 Hz, 3H), 9.11 (s, 1H), 8.53 (s, 1H), 8.42 (d, J = 8.8 Hz, 2H), 7.15 (d, J = 8.9 Hz, 2H), 6.67 (d, J = 2.2 Hz, 2H), 6.30 (t, J = 2.1 Hz, 1H), 3.88 (s, 3H). [M + H]⁺: 365.0. |
| | (E)-N′-(3,5- | |
| | dihydroxybenzylidene)-6- | |
| | (4- | |
| | methoxyphenyl)pyrazine- | |
| | 2-carbohydrazide | |
| 21 | 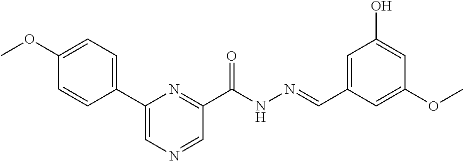 | ¹H-NMR, 400 MHz, DMSO-d₆ δ = 12.01 (br s, 1H), 9.73 (br s, 1H), 9.47 (s, 1H), 9.11 (s, 1H), 8.59 (s, 1H), 8.48-8.37 (m, 2H), 7.19-7.11 (m, 2H), 6.85 (d, J = 1.4 Hz, 1H), 6.76 (s, 1H), 6.44 (t, J = 2.2 Hz, 1H), 3.88 (s, 3H), 3.77 (s, 3H). [M + H]⁺: 379.1. |
| | (E)-N′-(3-hydroxy-5- | |
| | methoxybenzylidene)-6- | |
| | (4- | |
| | methoxyphenyl)pyrazine- | |
| | 2-carbohydrazide | |
| 22 | 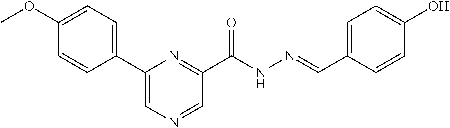 [SMILES: COc1ccc(-c2cncc(C(=O)N/N=C/c3ccc(O)cc3)n2)cc1] | ¹H-NMR, 400 MHz, DMSO-d₆ δ = 11.88 (s, 1H), 10.00 (s, 1H), 9.46 (s, 1H), 9.10 (s, 1H), 8.61 (s, 1H), 8.42 (d, J = 8.9 Hz, 2H), 7.63 (d, J = 8.7 Hz, 2H), 7.14 (d, J = 8.9 Hz, 2H), 6.88 (d, J = 8.6 Hz, 2H), 3.88 (s, 3H) [M + H]⁺: 349.1. |
| | (E)-N′-(4- | |
| | hydroxybenzylidene)-6- | |
| | (4- | |
| | methoxyphenyl)pyrazine- | |
| | 2-carbohydrazide | |
| 23 | 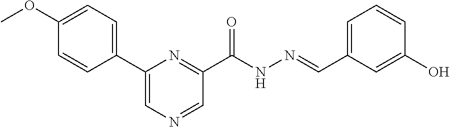 [SMILES: COc1ccc(-c2cncc(C(=O)N/N=C/c3cccc(O)c3)n2)cc1] | ¹H-NMR, 400 MHz, DMSO-d₆ δ = 12.01 (br s, 1H), 9.69 (br s, 1H), 9.47 (s, 1H), 9.11 (s, 1H), 8.64 (s, 1H), 8.42 (d, J = 8.8 Hz, 2H), 7.34-7.24 (m, 2H), 7.21-7.10 (m, 3H), 6.87 (dd, J = 2.1, 7.8 Hz, 1H), 3.88 (s, 3H). [M + H]⁺: 349.1. |
| | (E)-N′-(3- | |
| | hydroxybenzylidene)-6- | |
| | (4- | |
| | methoxyphenyl)pyrazine- | |
| | 2-carbohydrazide | |
| 24 | 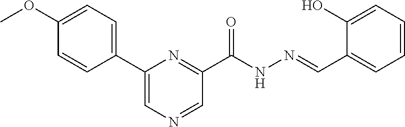 [SMILES: COc1ccc(-c2cncc(C(=O)N/N=C/c3ccccc3O)n2)cc1] | ¹H-NMR, 400 MHz, DMSO-d₆ δ = 12.33 (br s, 1H), 11.21 (br s, 1H), 9.48 (s, 1H), 9.13 (s, 1H), 8.95 (s, 1H), 8.49-8.38 (m, 2H), 7.61 (dd, J = 1.5, 7.7 Hz, 1H), 7.39-7.30 (m, 1H), 7.16 (d, J = 8.8 Hz, 2H), 7.03-6.91 (m, 2H), 3.88 (s, 3H). [M + H]⁺: 349.1. |
| | (E)-N′-(2- | |
| | hydroxybenzylidene)-6- | |
| | (4- | |
| | methoxyphenyl)pyrazine- | |
| | 2-carbohydrazide | |
| 25 | 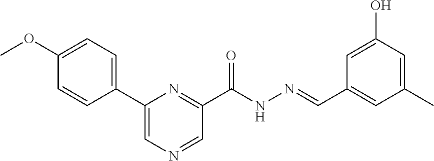 | ¹H-NMR, 400 MHz, DMSO-d₆ δ = 11.99 (br s, 1H), 10.05 (br s, 1H), 9.41 (s, 1H), 9.07 (s, 1H), 8.64 (s, 1H), 8.31 (br d, J = 8.7 Hz, 2H), 7.40 (s, 2H), 7.12 (s, 1H), 6.96 (br d, J = 8.7 Hz, 2H), 2.35 (s, 6H). [M + H]⁺: 363.1. |
| | (E)-N′-(3-hydroxy-5- | |
| | methylbenzylidene)-6-(4- | |
| | methoxyphenyl)pyrazine- | |
| | 2-carbohydrazide | |
| 26 | 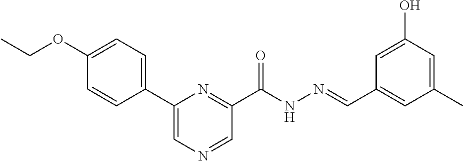 [SMILES: CCOc1ccc(-c2cncc(C(=O)N/N=C/c3cc(C)cc(O)c3)n2)cc1] | ¹H-NMR, 400 MHz, DMSO-d₆ δ = 11.99 (s, 1H), 9.56 (s, 1H), 9.46 (s, 1H), 9.10 (s, 1H), 8.59 (s, 1H), 8.40 (d, J = 8.8 Hz, 2H), 7.13 (d, J = 8.9 Hz, 2H), 7.02 (d, J = 16.1 Hz, 2H), 6.69 (s, 1H), 4.16 (q, J = 7.0 Hz, 2H), 2.29 (s, 3H), 1.39 (t, J = 6.9 Hz, 3H). [M + H]⁺: 377.1. |
| | (E)-6-(4-ethoxyphenyl)- | |
| | N′-(3-hydroxy-5- | |
| | methylbenzylidene)pyrazine- | |
| | 2-carbohydrazide | |
| 27 | 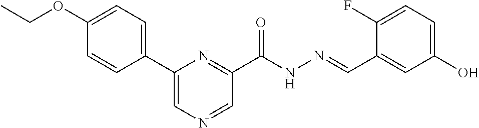 [SMILES: CCOc1ccc(-c2cncc(C(=O)N/N=C/c3cc(O)ccc3F)n2)cc1] | ¹H-NMR, 400 MHz, DMSO-d₆ δ = 12.22 (s, 1H), 9.68 (s, 1H), 9.48 (s, 1H), 9.12 (s, 1H), 8.92 (s, 1H), 8.42 (br d, J = 8.8 Hz, 2H), 7.41 (dd, J = 3.1, 5.7 Hz, 1H), 7.21-7.08 (m, 3H), 6.93- 6.82 (m, 1H), 4.15 (q, J = 6.8 Hz, 2H), 1.39 (t, J = 7.0 Hz, 3H). [M + H]⁺: 381.1. |
| | (E)-6-(4-ethoxyphenyl)- | |
| | N′-(2-fluoro-5- | |
| | hydroxybenzylidene)pyrazine- | |
| | 2-carbohydrazide | |
| 28 | 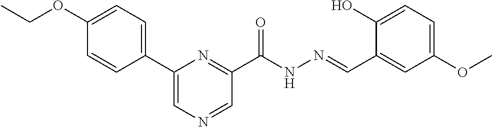 [SMILES: CCOc1ccc(-c2cncc(C(=O)N/N=C/c3cc(OC)ccc3O)n2)cc1] | ¹H-NMR, 400 MHz, DMSO-d₆ δ = 12.28 (s, 1H), 10.59 (s, 1H), 9.46 (s, 1H), 9.10 (s, 1H), 8.92 (s, 1H), 8.41 (d, J = 8.9 Hz, 2H), 7.20-7.08 (m, 3H), 6.98-6.86 (m, 2H), 4.15 (q, J = 7.0 Hz, 2H), 3.75 (s, 3H), 1.38 (t, J = 6.9 Hz, 3H). [M + H]⁺: 393.3. |
| | (E)-6-(4-ethoxyphenyl)- | |
| | N′-(2-hydroxy-5- | |
| | methoxybenzylidene)pyrazine- | |
| | 2-carbohydrazide | |
| 29 | 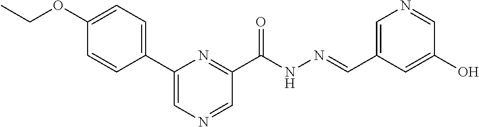 [SMILES: CCOc1ccc(-c2cncc(C(=O)N/N=C/c3cncc(O)c3)n2)cc1] | ¹H-NMR, 400 MHz, DMSO-d₆ δ = 12.19 (s, 1H), 10.39-10.22 (m, 1H), 9.46 (s, 1H), 9.11 (s, 1H), 8.75-8.70 (m, 1H), 8.40 (d, J = 8.9 Hz, 2H), 8.35 (d, J = 1.6 Hz, 1H), 8.22 (d, J = 2.8 Hz, 1H), 7.72-7.56 (m, 1H), 7.12 (d, J = 8.9 Hz, 2H), 4.15 (q, J = 7.0 Hz, 2H), 1.38 (t, J = 6.9 Hz, 3H). [M + H]⁺: 364.4. |
| | (E)-6-(4-ethoxyphenyl)- | |
| | N′-((5-hydroxypyridine- | |
| | 3-yl)methylene)pyrazine- | |
| | 2-carbohydrazide | |
| 30 | 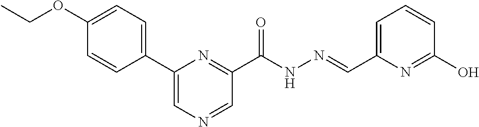 [SMILES: CCOc1ccc(-c2cncc(C(=O)N/N=C/c3cccc(O)n3)n2)cc1] | ¹H-NMR, 400 MHz, DMSO-d₆ δ = 12.47 (s, 1H), 11.14 (brs, 1H), 9.47 (s, 1H), 9.11 (s, 1H), 8.53 (s, 1H), 8.40 (d, J = 8.8 Hz, 2H), 7.57 (t, J = 8 Hz, 1H), 7.11 (d, J = 8 Hz, 2H), 6.80 (brs, 1H), 6.51 (d, J = 8 Hz, 1H), 4.17-4.15, 4.17- 4.11 (q, J = 8 Hz, 2H), 1.37 (t, J = 8 Hz, 3H). [M + H]⁺: 362.1. |
| | (E)-6-(4-ethoxyphenyl)- | |
| | N′-((6-hydroxypyridine- | |
| | 2-yl)methylene)pyrazine- | |
| | 2-carbohydrazide | |
| 31 | 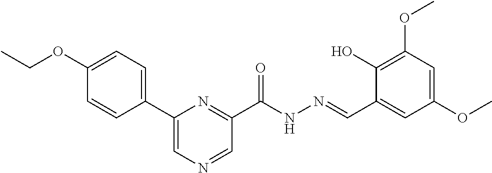 [SMILES: CCOc1ccc(-c2cncc(C(=O)N/N=C/c3cc(OC)cc(OC)c3O)n2)cc1] | ¹H-NMR, 400 MHz, DMSO-d₆ δ = 12.24 (br s, 1H), 10.08 (s, 1H), 9.46 (s, 1H), 9.10 (s, 1H), 8.95 (s, 1H), 8.41 (d, J = 8.8 Hz, 2H), 7.17- 7.08 (m, 2H), 6.79-6.64 (m, 2H), 4.14 (q, J = 7.0 Hz, 2H), 3.87-3.70 (m, 6H), 1.38 (t, J = 7.0 Hz, 3H). [M + H]⁺: 423.2. |
| | (E)-6-(4-ethoxyphenyl)- | |
| | N′-(2-hydroxy-3,5- | |
| | dimethoxybenzylidene)pyrazine- | |
| | 2-carbohydrazide | |
| 32 | 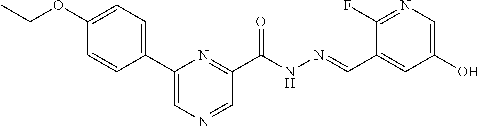 [SMILES: CCOc1ccc(-c2cncc(C(=O)N/N=C/c3cc(O)cnc3F)n2)cc1] | ¹H-NMR, 400 MHz, DMSO-d₆ δ = 12.32 (s, 1H), 10.25 (s, 1H), 9.47 (s, 1H), 9.11 (s, 1H), 8.84 (s, 1H), 8.40 (d, J = 8.9 Hz, 2H), 7.90-7.74 (m, 2H), 7.12 (d, J = 8.8 Hz, 2H), 4.14 (q, J = 6.9 Hz, 2H), 1.38 (t, J = 6.9 Hz, 3H). [M + H]⁺: 382.4. |
| | (E)-6-(4-ethoxyphenyl)- | |
| | N′-((2-fluoro-5- | |
| | hydroxypyridine-3- | |
| | yl)methylene)pyrazine-2- | |
| | carbohydrazide | |

| | |
|---|---|
| 33 | 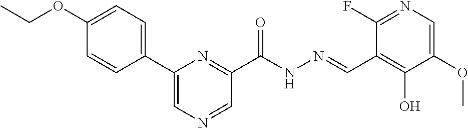 [SMILES: CCOc1ccc(-c2cncc(C(=O)N/N=C/c3c(F)ncc(OC)c3O)n2)cc1] |
| | (E)-6-(4-ethoxyphenyl)-N′-((2-fluoro-4-hydroxy-5- |
| | methoxypyridine-3-yl)methylene)pyrazine-2- |
| | carbohydrazide |
| 34 | 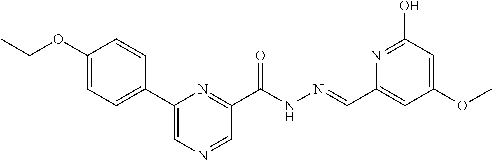 [SMILES: CCOc1ccc(-c2cncc(C(=O)N/N=C/c3cc(OC)cc(O)n3)n2)cc1] |
| | (E)-6-(4-ethoxyphenyl)-N′-((6-hydroxy-4- |
| | methoxypyridine-2-yl)methylene)pyrazine-2- |
| | carbohydrazide |
| 35 | 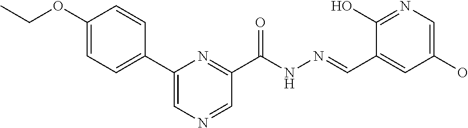 [SMILES: CCOc1ccc(-c2cncc(C(=O)N/N=C/c3cc(O)cnc3O)n2)cc1] |
| | (E)-6-(4-ethoxyphenyl)-N′-((2-hydroxy-5- |
| | methoxypyridine-3-yl)methylene)pyrazine-2- |
| | carbohydrazide |
| 36 | 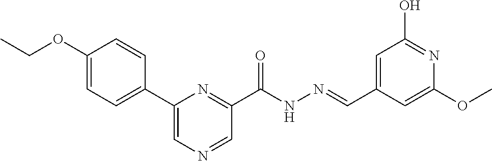 [SMILES: CCOc1ccc(-c2cncc(C(=O)N/N=C/c3cc(O)nc(OC)c3)n2)cc1] |
| | (E)-6-(4-ethoxyphenyl)-N′-((2-hydroxy-6- |
| | methoxypyridine-4-yl)methylene)pyrazine-2- |
| | carbohydrazide |

### Example 3. Tests In Vitro

Biological assays were performed on Chinese hamster ovary (CHO) cells that express human sodium channels Nav 1.8 or Nav 1.7 in a stable manner.

The experimental protocol voltage-clamp using the whole cell configuration was established on the automated ScreenPatch® 384P platform (SP384PE, Nanion Technologies, Livingston, NJ) and the results were recorded with a Nanion 384-well Patch Clamp chip (NPC) (Nanion Technologies, Livingston, NJ).

Formula I compounds were diluted in eight concentrations in the extracellular solution composed of physiological saline solution, buffered with HEPES (mM): NaCl, 137; KCl, 4; CaCl₂, 3.8; MgCl₂, 1; HEPES, 10; Glucose, 10; pH 7.4. The extracellular solution is composed of (mM) CsCl, 50; CsF, 90; MgCl₂, 5; EGTA, 5; HEPES, 10; pH 7.2. The duration of exposure of each compound with cells expressing Nav 1.7 or Nav 1.8 was at least five minutes, and the assays were performed at room temperature.

The measurements of the sodium currents of Nav 1.8 and Nav 1.7 were obtained using the voltage protocol described below.

A holding voltage of −100 mV was established followed by an inactivation voltage step at −40 mV for 8 seconds, followed by a step of −100 mV for 20 ms, followed by a 20 ms step for 10 mV for Nav 1.8 or 0 mV for Nav 1.7 (TP1A) before returning to the holding voltage of −100 mV.

The protocol was repeated at a frequency of 0.05 Hz and the amplitude of the current was quantified throughout the recording of the TP1A phase. The variation in the peak current amplitude was evaluated according to the Formula described below, after exposing the cells expressing the channels to each concentration of the different compounds:

$$\% Block = ( 1 - ( {I}_{TP 1 A , molecule} / {I}_{TP 1 A , basal} ) \times 100 \% ,$$

wherein I_{T1KA,basal} and I_{TP1A,molecule} represent the peaks of sodium current input into TP1A before exposure to the compound and in the presence of the compound, respectively.

The decrease in the peak current amplitude, after the exposure of the cells to the compounds, was used to calculate the percentage of relative blockage of the channels in relation to the positive control according to the Formula below:

$$\% {Block}^{′} = 100 \% - ( ( \% Block - \% CP ) * ( 100 \% / ( \% V - \% CP ) ) ,$$

where % V and % CP represent the means of the values of current inhibition with a vehicle (DMSO) and positive controls, respectively. The sodium currents of the positive control were considered as 100%:

$$\% {Block}^{′} = ( 100 \% / [ 1 + {( [ Test ] / {CI}_{50} )}^{N} ] ,$$

wherein [Test] represents the concentration of the molecule evaluated, IC₅₀ is the concentration of the compound that generates half of the maximum inhibition, N is Hill's coefficient, and % Blockage' is the percentage of sodium channel current (Nav 1.8 and Nav 1.7) inhibited at each concentration of the molecule evaluated. Data were obtained by nonlinear regression (nonlinear least squares) with XLfit for Excel (Microsoft, Redmond, WA).

The compounds were tested in at least one assay to obtain the value of CI₅₀. For compounds that were tested in two or more assays, the results are described as the means of the CI₅₀ values.

Table 4 shows the efficacy in vitro of selected compounds against Nav1.8 and Nav1.7. CI₅₀ values less than 500 nM are represented with the legend (+++); CI₅₀ values between 500 nM and 1000 nM are represented with the legend (++), CI₅₀ values greater than 1000 nM are demonstrated with the symbology (+).

TABLE 4
CI₅₀ VALUES OF THE COMPOUNDS
OF THIS INVENTION IN NAV 1.8
AND NAV 1.7 CHANNELS.

| | Nav1.8 | Nav1.7 |
|---|---|---|
| Compound | IC₅₀ | IC₅₀ |
| No | (nM) | (nM) |
| 1 | + | + |
| 2 | + | + |
| 3 | + | + |
| 4 | + | + |
| 5 | +++ | + |
| 6 | + | + |
| 7 | +++ | + |
| 8 | ++ | + |
| 9 | +++ | + |
| 12 | + | + |
| 13 | + | + |
| 14 | + | + |
| 15 | + | + |
| 16 | + | + |
| 18 | + | + |
| 19 | + | + |
| 20 | + | + |
| 21 | + | + |
| 22 | + | + |
| 23 | + | + |
| 24 | + | + |
| 25 | ++ | + |
| 26 | +++ | + |
| 27 | ++ | + |
| 28 | +++ | + |
| 29 | + | + |

From these results, the blocking activity of Nav 1.7 and/or 1.8 is proven, whose application is readily performed by those skilled in the art in pharmaceutical compositions, which may comprise one or more of the aforementioned Formula I compounds, kits, in addition to uses in the treatment of pain-related pathologies.

In particular, these results indicate the possibility of using Formula (I) compounds in the preparation of drugs for the treatment of conditions such as peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

It should be understood that the embodiments described above are merely illustrative and that several modifications can be made by a person skilled in the art without departing from the scope of this invention. Consequently, the present invention should not be regarded as being limited to the illustrative specifications described in this application.

1. A compound of Formula (I) 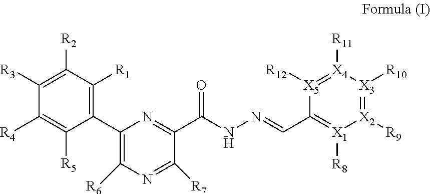
or a pharmaceutically acceptable salt, hydrate, solvate and isomer thereof, wherein:
one of X1, X2, X3, X4 and X5 is nitrogen and the others are carbon, where when one of X1, X2, X3, X4 and X5 is nitrogen, the corresponding R (R5, R6 and/or R7) will be null;
R1, R2, R3, R4, and R5 are independently selected from the group consisting of hydrogen, halogen, hydroxy, alkoxy C1-C6 linear or branched, alkyl C1-C6 linear or branched, wherein at least one of R1-R5 is hydroxy, when R6-R12 are different from hydroxy;
R6 and R7 are independently selected from the group consisting of hydrogen, halogen, hydroxy, linear or branched C1-C6 alkoxy, C1-C6 linear or branched alkyl, wherein at least one of R6-R7 is hydroxy, when R1-R5 and R8-R12 are different from hydroxy; and
R8, R9, R10, R11, and R12 are independently selected from the group consisting of hydrogen, linear or branched C1-C6 alkoxy, alkyl linear or branched C1-C6; halogen, hydroxy, wherein at least one of R8-R12 is hydroxy, while R1-R7 are different from hydroxy.

2. The compound, according to claim 1, characterized by being selected from the group consisting of:
(E)-N′-(3,5-dimethoxybenzylidene)-6-(4-hydroxyphenyl)pyrazine-2-carbohydrazide (Compound 1);
(E)-N′-(3,5-dimethylbenzylidene)-6-(4-hydroxyphenyl)pyrazine-2-carbohydrazide (Compound 2);
(E)-N′-(2-fluoro-5-methoxybenzylidene)-6-(4-hydroxyphenyl)pyrazine-2-carbohydrazide (Compound 3);
(E)-6-(4-hydroxyphenyl)-N′-((5-hydroxypyridine-3-yl)methylene)pyrazine-2-carbohydrazide (Compound 4);
(E)-N′-(3,5-dimethoxybenzylidene)-6-(2-hydroxy-4-methoxyphenyl)pyrazine-2-carbohydrazide (Compound 5);
(E)-N′-(3,5-dimethoxybenzylidene)-6-(3-hydroxy-4-methoxyphenyl)pyrazine-2-carbohydrazide (Compound 6);
(E)-N′-(3,5-dimethylbenzylidene)-6-(2-hydroxy-4-methoxyphenyl)pyrazine-2-carbohydrazide (Compound 7);
(E)-6-(4-ethoxy-2-hydroxyphenyl)-N′-(2-fluoro-5-methoxybenzylidene)pyrazine-2-carbohydrazide (Compound 8);
(E)-6-(4-ethoxy-2-hydroxyphenyl)-N′-((2-fluoro-5-methoxypyridine-3-yl)methylene)pyrazine-2-carbohydrazide (Compound 9);
(E)-6-(4-ethoxy-3-hydroxyphenyl)-N′-((2-fluoro-5-methoxypyridine-3-yl)methylene)pyrazine-2-carbohydrazide (Compound 10);
(E)-6-(4-ethoxy-3-hydroxyphenyl)-N′-(2-fluoro-5-methoxybenzylidene)pyrazine-2-carbohydrazide (Compound 11);
(E)-N′-(3,5-dimethoxybenzylidene)-5-hydroxy-6-(4-methoxyphenyl)pyrazine-2-carbohydrazide (Compound 12);
(E)-N′-(3,5-dimethylbenzylidene)-5-hydroxy-6-(4-methoxyphenyl)pyrazine-2-carbohydrazide (Compound 13);
(E)-N′-(3,5-dimethoxybenzylidene)-3-hydroxy-6-(4-methoxyphenyl)pyrazine-2-carbohydrazide (Compound 14);
(E)-N′-(3,5-dimethylbenzylidene)-3-hydroxy-6-(4-methoxyphenyl)pyrazine-2-carbohydrazide (Compound 15);
(E)-6-(4-ethoxyphenyl)-N′-((2-fluoro-5-methoxypyridine-3-yl)methylene)-5-hydroxypyrazine-2-carbohydrazide (Compound 16);
(E)-6-(4-ethoxyphenyl)-N′-((2-fluoro-5-methoxypyridine-3-yl)methylene)-3-hydroxypyrazine-2-carbohydrazide (Compound 17);
(E)-6-(4-ethoxy-2-fluorophenyl)-5-hydroxy-N′-(3-methoxybenzylidene)pyrazine-2-carbohydrazide (Compound 18);
(E)-6-(4-ethoxy-2-fluorophenyl)-3-hydroxy-N′-(3-methoxybenzylidene)pyrazine-2-carbohydrazide (Compound 19);
(E)-N′-(3,5-dihydroxybenzylidene)-6-(4-methoxyphenyl)pyrazine-2-carbohydrazide (Compound 20);
(E)-N′-(3-hydroxy-5-methoxybenzylidene)-6-(4-methoxyphenyl)pyrazine-2-carbohydrazide (Compound 21);
(E)-N′-(4-hydroxybenzylidene)-6-(4-methoxyphenyl)pyrazine-2-carbohydrazide (Compound 22);
(E)-N′-(3-hydroxybenzylidene)-6-(4-methoxyphenyl)pyrazine-2-carbohydrazide (Compound 23);
(E)-N′-(2-hydroxybenzylidene)-6-(4-methoxyphenyl)pyrazine-2-carbohydrazide (Compound 24);
(E)-N′-(3-hydroxy-5-methylbenzylidene)-6-(4-methoxyphenyl)pyrazine-2-carbohydrazide (Compound 25);
(E)-6-(4-ethoxyphenyl)-N′-(3-hydroxy-5-methylbenzylidene)pyrazine-2-carbohydrazide (Compound 26);
(E)-6-(4-ethoxyphenyl)-N′-(2-fluoro-5-hydroxybenzylidene)pyrazine-2-carbohydrazide (Compound 27);
(E)-6-(4-ethoxyphenyl)-N′-(2-hydroxy-5-methoxybenzylidene)pyrazine-2-carbohydrazide (Compound 28);
(E)-6-(4-ethoxyphenyl)-N′-((5-hydroxypyridin-3-yl)methylene)pyrazine-2-carbohydrazide (Compound 29);
(E)-6-(4-ethoxyphenyl)-N′-((6-hydroxypyridin-2-yl)methylene)pyrazine-2-carbohydrazide (Compound 30);
(E)-6-(4-ethoxyphenyl)-N′-(2-hydroxy-3,5-dimethoxybenzylidene)pyrazine-2-carbohydrazide (Compound 31);
(E)-6-(4-ethoxyphenyl)-N′-((2-fluoro-5-hydroxypyridine-3-yl)methylene)pyrazine-2-carbohydrazide (Compound 32);
(E)-6-(4-ethoxyphenyl)-N′-((2-fluoro-4-hydroxy-5-methoxypyridine-3-yl)methylene)pyrazine-2-carbohydrazide (Compound 33);
(E)-6-(4-ethoxyphenyl)-N′-((6-hydroxy-4-methoxypyridine-2-yl)methylene)pyrazine-2-carbohydrazide (Compound 34);
(E)-6-(4-ethoxyphenyl)-N′-((2-hydroxy-5-methoxypyridine-3-yl)methylene)pyrazine-2-carbohydrazide (Compound 35); and
(E)-6-(4-ethoxyphenyl)-N′-((2-hydroxy-6-methoxypyridine-4-yl)methylene)pyrazine-2-carbohydrazide (Compound 36).

3. The compound according to claim 1, characterized by blocking voltage-gated sodium channels Nav 1.7 and/or Nav 1.8.

4. A pharmaceutical composition characterized by comprising a therapeutically effective amount of one or more compounds of Formula (I) or of a pharmaceutically acceptable salt, hydrate, solvate, and isomer thereof, as defined in claim 1, and one or more pharmaceutically acceptable excipients.

5. The pharmaceutical composition according to claim 4, characterized by being formulated as an oral, sublingual, nasal, parenteral, injectable, submuscular, topical, transdermal, ocular, or rectal composition.

6.-7. (canceled)

8. A method of treating, preventing, relieving, suppressing, and/or controlling pathologies related to neuropathic pain characterized by the administration of an effective amount of at least one compound of Formula (I) or a pharmaceutically acceptable salt, hydrate, solvate and isomer thereof, as defined in claim 1.

9. The method according to claim 8, characterized by being for the treatment or prophylaxis of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

10. The method according to claim 8, characterized by the administration of at least one compound of Formula (I) being selected from the group comprising oral, sublingual, nasal, parenteral, injectable, submuscular, topical, transdermal, ocular, and rectal routes.

11. A process of making the compound of claim 1 comprising the steps:
(a) formation of the Formula III intermediate: 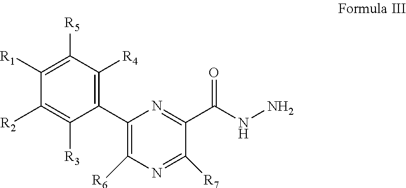
from the hydrazinolysis reaction of a Formula IV intermediate: 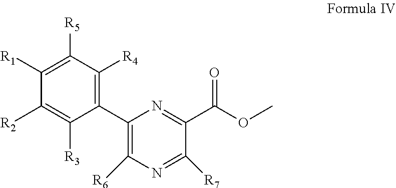
(b) obtaining Formula I compound; 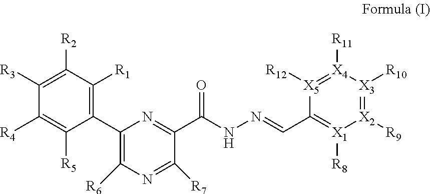
from the condensation of Formula II intermediates: 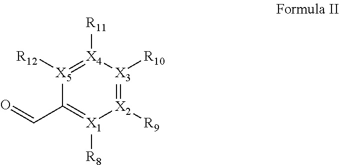
and Formula III, with or without the presence of a catalyst and a suitable solvent;
wherein,
one of X1, X2, X3, X4 and X5 is nitrogen and the others are carbon, wherein when one of X1, X2, X3, X4 and X5 is nitrogen, the corresponding R (R5, R6 and/or R7) will be null;
R1, R2, R3, R4, and R5 are independently selected from the group consisting of hydrogen, halogen, hydroxy, alkoxy C1-C6 linear or branched, alkyl C1-C6 linear or branched, wherein at least one of R1-R5 is hydroxy, when R6-R12 are different from hydroxy;
R6 and R7 are independently selected from the group consisting of hydrogen, halogen, hydroxy, linear or branched, C1-C6 alkyl, C1-C6 linear or branched alkoxy, wherein at least one of R6-R7 is hydroxy, when R1-R5 and R8-R12 are different from hydroxy; and
R8, R9, R10, R11, and R12 are independently selected from the group consisting of hydrogen, linear or branched C1-C6 alkoxy, alkyl linear or branched C1-C6, halogen, hydroxy, where at least one of R8-R12 is hydroxy, while R1-R7 are different from hydroxy.

12. The method according to claim 11, characterized by said catalyst of step (b) being selected from concentrated hydrochloric acid, acetic acid, trifluoroacetic acid, formic acid, or combinations thereof and said solvent being selected from dimethylformamide, dimethylsulfoxide, alcohols, or combinations thereof.

13. A kit comprising a pharmaceutical composition, as defined in claim 4, and an application device.
